(19) United States
(12) Patent Application Publication
(10) Pub. No.: US 2011/0142780 A1
(43) Pub. Date: Jun. 16, 2011

(54) AGENT FOR KERATIN-CONTAINING FIBERS, COMPRISING AT LEAST ONE PARTICULAR AMPHIPHILIC CATIONIC POLYMER AND AT LEAST ONE POLYMER WITH SILICON-CONTAINING SIDECHAINS AND ANIONIC GROUPS

(76) Inventors: Dirk Hentrich, Hamburg (DE); Sabine Albrechtsen, Elmshorn (DE); Esma Alanya, Hamburg (DE)

(21) Appl. No.: 13/030,188

(22) Filed: Feb. 18, 2011

Related U.S. Application Data
(63) Continuation of application No. PCT/EP2009/059356, filed on Jul. 21, 2009.

(30) Foreign Application Priority Data
Aug. 18, 2008 (DE) 10 2008 038 104.7

(51) Int. Cl. A61K 8/89; A61Q 5/00

(52) U.S. Cl. 424/70.19

(57) ABSTRACT

Agent for treating keratin-containing fibers, particularly, human hair, containing in a cosmetically-acceptable vehicle: (a) at least one amphiphilic, cationic polymer having at least one formula (I) structural unit, at least one formula (II) structural unit, at least one formula (III) structural, and at least one formula (IV) structural unit, where R¹ and R⁴ independently=H or methyl; X¹ and X² independently=O or NH, A¹ and A² independently=ethan-1,2-diyl, propan-1,3-diyl or butan-1,4-diyl, R², R³, R⁵ and R⁶ independently=(C₁-C₄) alkyl, R⁷=(C₈-C₃₀) alkyl and (b) at least one polymer having one or more silicon-containing sidechains and one or more anionic groups, use of the agent for temporary shaping of hair and for haircare, particularly in the form of a hair cream or hair gel.

### CROSS REFERENCE TO RELATED APPLICATIONS

The present application is a continuation of International Patent Application No. PCT/EP2009/059356 filed 21 Jul. 2009, which claims priority to German Patent Application No. 10 2008 038 104.7 filed 18 Aug. 2008, both of which are incorporated herein by reference.

The present invention relates to agents for treating hair comprising a combination of at least one specific amphiphilic, cationic polymer with at least one polymer containing silicone-containing side chains and anionic groups, use of these agents for temporary shaping and/or for care of keratin-containing fibers, and hair gels based on these agents.

Keratin-containing fibers include all animal hair (e.g., wool, horsehair, angora hair, furs, feathers and products or fabrics produced from them). However, keratinic fibers preferably concern human hair.

Today, a suitably looking hairstyle is generally regarded as an essential part of a well groomed appearance. Based on fashion trends, time and again hairstyles are considered chic which, for many types of hair, can only be formed or sustained over a period of up to several days by use of certain consolidating materials. Thus, hair treatments which provide a permanent or temporary hairstyling play an important role. Temporary styling intended to provide a good hold without compromising the healthy appearance of the hair, such as gloss, can be obtained, for example, by use of hairsprays, hair waxes, hair gels, hair foams, setting lotions, etc.

Suitable compositions for temporary hairstyling usually contain synthetic polymers as the styling component. Preparations containing a dissolved or dispersed polymer can be applied onto hair by propellants or by a pumping mechanism. Hair gels and hair waxes, however, are not generally applied directly on the hair, but rather dispersed with a comb or by hand.

An important property of an agent for temporary styling of keratin fibers, also referred to as styling agents, involves giving the treated fibers the strongest possible hold for the shape created. If the keratinic fibers are human hair, then one also speaks of a strong hairstyle hold or high degree of hold of the styling agent. Styling hold is determined by the type and quantity of synthetic polymer used; however, other components of the styling agent may also influence hold.

In addition to a high degree of hold, styling agents must fulfill a variety of additional requirements. These requirements can be broadly divided into hair properties, formulation properties (e.g., properties of the foam, gel or aerosol spray), and properties concerning the handling of the styling agent, with particular importance attached to the hair properties. These include moisture resistance, low stickiness and a balanced conditioning effect. Furthermore, a styling agent should be universally applicable for as many types of hair as possible.

In an attempt to meet these various requirements, various synthetic polymers have been developed and are being used in styling agents. These polymers can be divided into cationic, anionic, non-ionic and amphoteric film-forming and/or setting polymers. Ideally these polymers form a polymer film when applied to hair, imparting a strong hold to the hairstyle while also being sufficiently flexible so as to not to break under stress. If the polymer film is too brittle, film plaques develop (i.e., residues that are shed with movement of the hair and give the impression that the user of the respective styling agent has dandruff).

To develop styling agents that in combination have all the desired properties still presents problems. This is particularly true for the combination of strong hold and simple, uniform application onto the keratin-containing fibers. In order to impart a strong hold, the setting polymer has to adhere well to the keratin-containing fibers. Still, the cosmetic agent, particularly the keratin-containing fibers that have been treated with it, should not feel sticky. The former is especially important for application as foam or as a gel or cream.

Accordingly, the present invention provides an agent for temporary shaping of keratinic fibers having a high degree of hold and, in particular, having excellent handleability during application onto the keratin-containing fibers (i.e., it is not sticky and imparts a good hold to the keratin-containing fibers without a sticky film).

It has now been surprisingly found that this can be achieved by a combination of specific polymers. The polymer combination according to the invention brings about not only a strong hold but also easy handling, especially when applied as a gel, as the gel has a pasty consistence that nevertheless allows it to be easily dispersed and feels barely sticky.

Accordingly, a first subject matter of the present invention is agents for treating keratin-containing fibers, especially human hair, comprising in a cosmetically acceptable carrier
(a) at least one amphiphilic, cationic polymer having at least one structural unit of Formula (I), at least one structural unit of Formula (II), at least one structural unit of Formula (III) and at least one structural unit of Formula (IV),

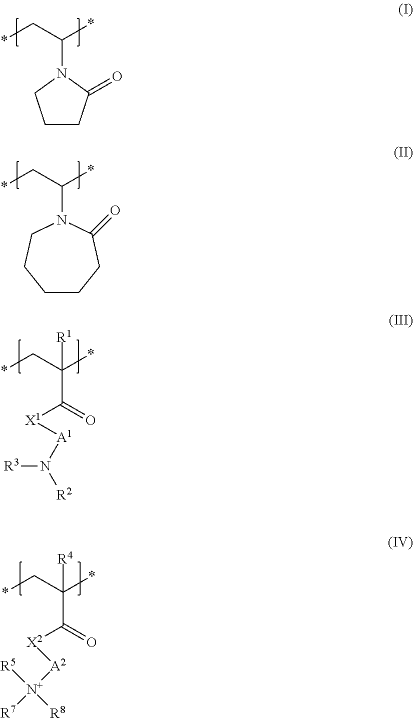

wherein
R¹ and R⁴ are, independently of one another, a hydrogen atom or a methyl group,
X¹ and X² are, independently of one another, for an oxygen atom or an NH group,
A¹ and A² are, independently of one another, an ethane-1,2-diyl, propane-1,3-diyl or butane-1,4-diyl group,
R², R³, R⁵ and R⁶ are, independently of one another, a (C₁ to C₄) alkyl group, and
R⁷ is a (C₈ to C₃₀) alkyl group, and
(b) at least one polymer having one or more silicon-containing side chains and one or more anionic groups.

In the above Formulae and all Formulae below, the symbol * represents a chemical bond that is a free valence of the corresponding structural fragment.

To compensate for the positive polymer charge, all possible physiologically acceptable anions can be used, such as chloride, bromide, hydrogen sulfate, methyl sulfate, ethyl sulfate, tetrafluoroborate, phosphate, hydrogen phosphate, dihydrogen phosphate or p-toluene sulfonate, or triflate.

Exemplary (C₁ to C₄) alkyl groups are methyl, ethyl, propyl, isopropyl, butyl, sec-butyl, isobutyl, tert-butyl.

Exemplary (C₈ to C₃₀) alkyl groups are octyl (capryl), decyl (caprinyl), dodecyl (lauryl), tetradecyl (myristyl), hexadecyl (cetyl), octadecyl (stearyl), eicosyl (arachyl), docosyl (behenyl).

Properties of the agent have proven to be particularly advantageous when it is in the form of a gel. This preferred packaging form is described later in detail.

The following amphiphilic, cationic polymers are preferably used in agents according to the invention when the amphiphilic, cationic polymers fulfill one or more of the following criteria:
R¹ and R⁴ are each a methyl group,
X¹ is an NH group,
X² is an NH group,
A¹ and A² are, independently of one another, ethane-1,2-diyl or propane-1,3-diyl,
R², R³, R⁵ and R⁶ are, independently of one another, methyl or ethyl, (preferably methyl),
R⁷ is a (C₁₀ to C₂₄) alkyl group, especially decyl (caprinyl), dodecyl (lauryl), tetradecyl (myristyl), hexadecyl (cetyl), octadecyl (stearyl), eicosyl (arachyl) or docosyl (behenyl).

Preferably, the structural unit of Formula (III) is chosen from at least one of the structural units of Formula (III-1) to (III-8)

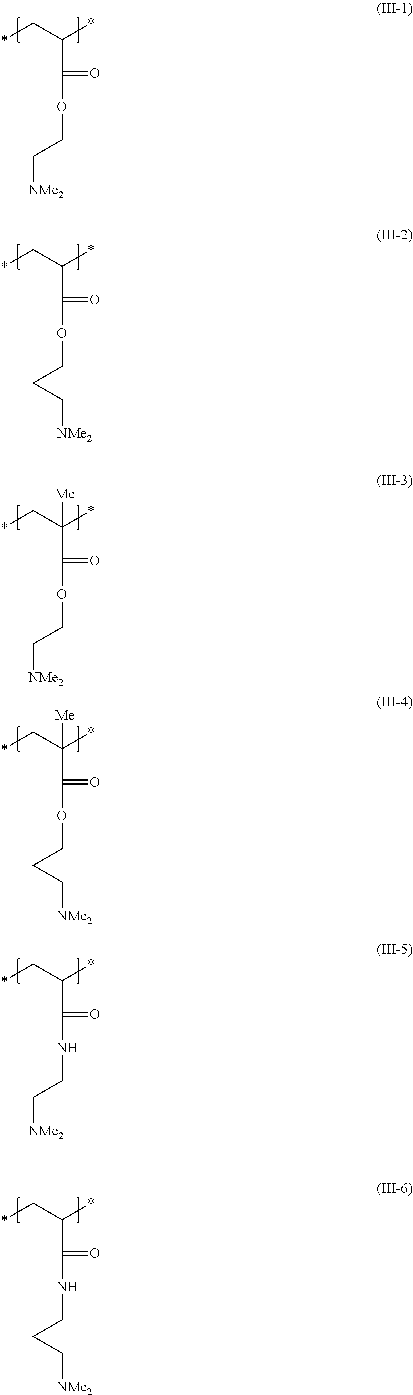

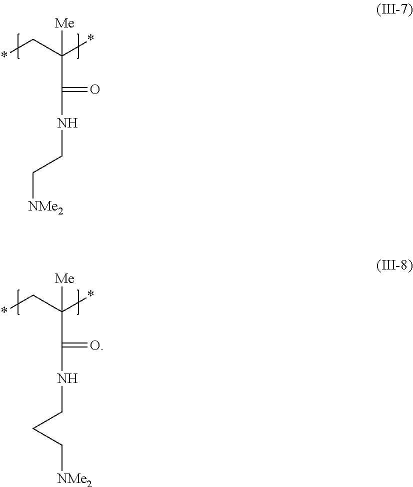

Even more preferably, the structural unit according to Formula (III-7) and/or of Formula (III-8) is chosen as the structural unit of Formula (III). According to the invention, the structural unit of Formula (III-8) is a quite particularly preferred structural unit.

Furthermore, the structural unit of Formula (IV) is preferably chosen from at least one structural unit of Formulas (IV-1) to (IV-8)

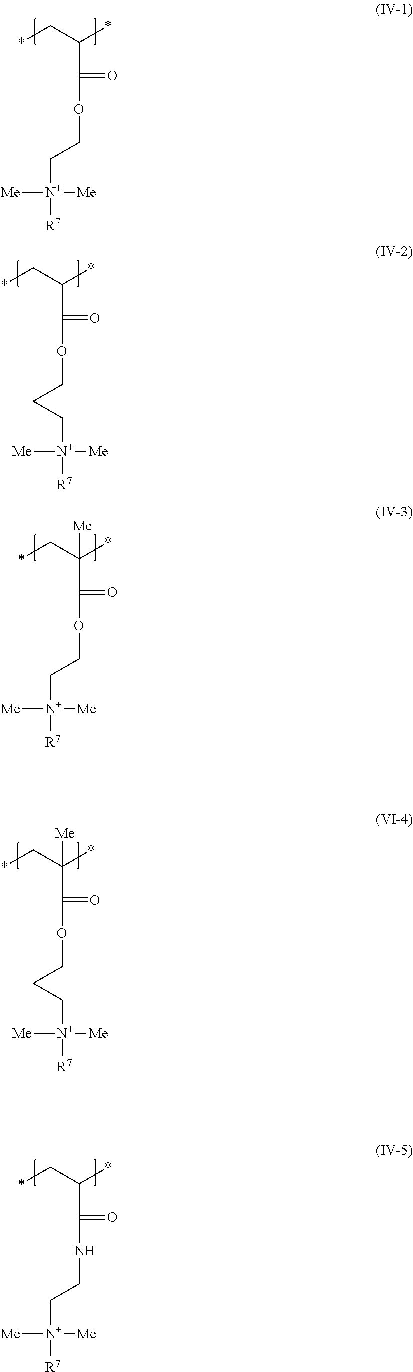

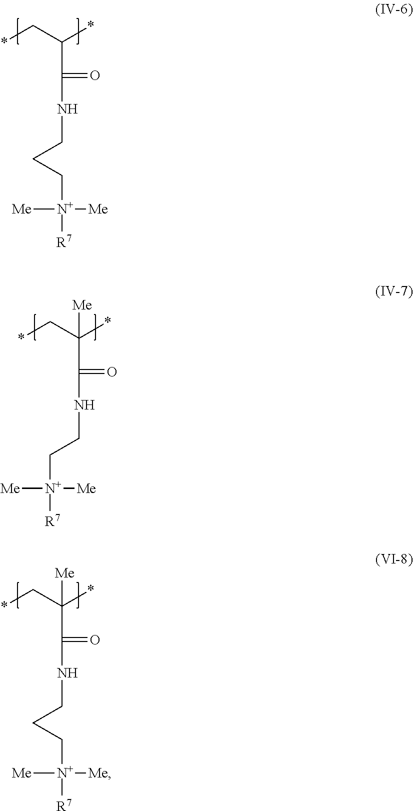

wherein each R⁷ is a (C₈ to C₃₀) alkyl group.

Structural units of Formula (IV-7) and/or of Formula (IV-8) are once again particularly preferred as the structural unit of Formula (IV), wherein each R⁷ is octyl (capryl), decyl (caprinyl), dodecyl (lauryl), tetradecyl (myristyl), hexadecyl (cetyl), octadecyl (stearyl), eicosyl (arachyl) or docosyl (behenyl). According to the invention, the structural unit of Formula (IV-8) is a quite particularly preferred structural unit of Formula (IV).

A quite particularly preferred amphiphilic, cationic polymer contains at least one structural unit of Formula (I), at least one structural unit of Formula (II), at least one structural unit of Formula (III-8), and at least one structural unit of Formula (IV-8),

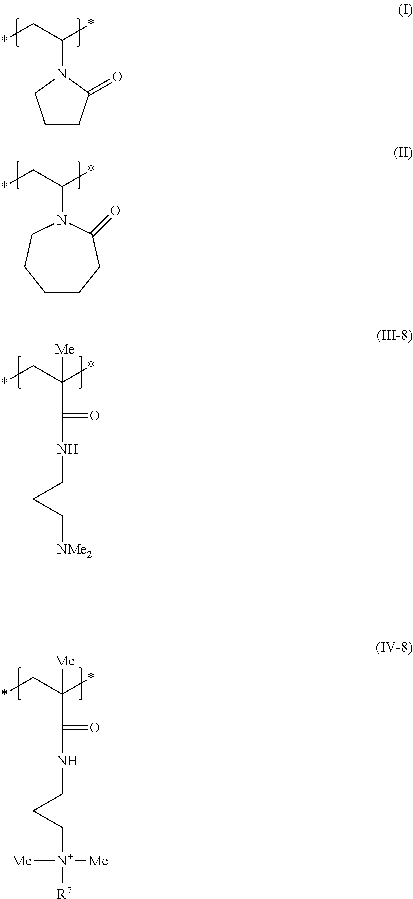

wherein R⁷ is a (Cₛ to C₃₀) alkyl group (especially octyl (capryl), decyl (caprinyl), dodecyl (lauryl), tetradecyl (myristyl), hexadecyl (cetyl), octadecyl (stearyl), eicosyl (arachyl), or docosyl (behenyl)).

Molecular weights of amphiphilic, cationic polymers according to the invention are preferably from 10,000 g/mol to 50,000,000 g/mol, more preferably from 50,000 g/mol to 5,000,000 g/mol, particularly preferably from 75,000 g/mol to 1,000,000 g/mol.

A particularly preferred amphiphilic, cationic polymer according to the invention is the copolymer of N-vinyl pyrrolidone, N-vinyl caprolactam, N-(3-dimethylaminopropyl)methacrylamide and 3-(methacryloylamino)propyl-lauryl-dimethylammonium chloride (INCI name: Polyquaternium-69), marketed, for example, under the trade name AquaStyle® 300 (28-32 wt. % active substance in an ethanol-water mixture) by the ISP company.

According to the invention, preferred agents contain the above amphiphilic, cationic polymers in an amount of 0.05 wt. % to 10.0 wt. %, preferably 0.05 wt. % to 5.0 wt. %, more preferably 0.1 wt. % to 4.0 wt. %, based on total weight of the agent.

Moreover, the agent contains, in addition to the previously defined amphiphilic, cationic polymer, at least one anionic polymer with silicone-containing side chains.

According to the invention, preferred agents contain polymers with silicone-containing side chains and anionic groups in an amount of 0.05 wt. % to 10.0 wt. %, preferably 0.05 wt. % to 5.0 wt. %, and more preferably 0.1 wt. % to 2.0 wt. %, based on total weight of the agent.

Polymers (a) and (b) are preferably used in a weight ratio of 5 to 1 to 1 to 5, especially 4 to 1 to 1 to 4.

Preferably, the silicone-containing side chains of the anionic polymers are chosen from representatives of the group of silicone copolyols. Silicone copolyols are silicones that have in their structure at least one unit of one or more alkylene glycols, especially ethylene glycol and/or propylene glycol.

The polymer having at least one silicone-containing side chain and at least one anionic group (hereinafter called polymer (b)) is preferably chosen from at least one polymer obtained by copolymerizing monomers (i) and optionally (ii)—
at least one monomer having an olefinic unsaturated group, a silicone group and, where no additional anionic monomer (ii) is added, at least one anionic group, and
optionally at least one additional monomer chosen from non-ionic monomers, anionic monomers, cationic monomers, associative monomers or crosslinking monomers.

At least one polymer (b) obtained by copolymerizing at least monomers (i) to (v) below is particularly suitable for use in the agent
(i) at least one monomer that carries an olefinic unsaturated group and a silicone group,
(ii) at least one additional monomer chosen from (preferably olefinic unsaturated) anionic monomers,
(iii) at least one additional non-ionic monomer,
(iv) optionally, at least one additional associative monomer, and
(v) optionally, at least one additional crosslinking monomer.

A preferred variant of polymer (b) is obtained by copolymerizing at least the following monomers (i) to (v)
(i) at least one monomer having an olefinic unsaturated group and a silicone group,
(ii) at least one additional monomer chosen from (preferably olefinic unsaturated) anionic monomers,
(iii) at least one additional non-ionic monomer,
(iv) optionally at least one additional associative monomer, and
(v) optionally at least one additional crosslinking monomer.

In the abovementioned embodiments, at least one monomer chosen from compounds of Formulae (A1) and (A2) is used

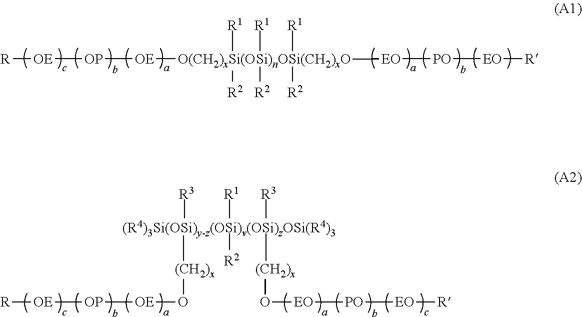

wherein
R¹ and R² are, independently of one another, a (C₁ to C₃₀) alkyl group, a (C₁ to C₂₀) alkyl group substituted with at least one halogen atom (e.g., —CCI₃, —CBr₃, —CF₃), a (C₃ to C₈) cycloalkyl group, a (C₆ to C₁₄) aryl group or (C₂ to C₂₀) alkenyl group,
R³ is a (C₁ to C₃₀) alkyl group, a (C₁ to C₂₀) alkyl group substituted with at least one halogen atom (e.g., —CCI₃, —CF₃), a (C₃ to Cg) cycloalkyl group, a (C₆ to C₁₄) aryl group or (C₂ to C₂₀) alkenyl group,
R⁴ are each independently a (C₁ to C₃₀) alkyl group, a (C₆ to C₁₄) aryl group, a (C₂ to C₂₀) alkenyl group,
R and R′ at least one of R or R′ is an ester group of an olefinic unsaturated carboxylic acid according to one of the Formulae (Est1) to (Est7), wherein the other is one of these ester groups of Formulae (Est1) to (Est7) or is a hydrogen atom,

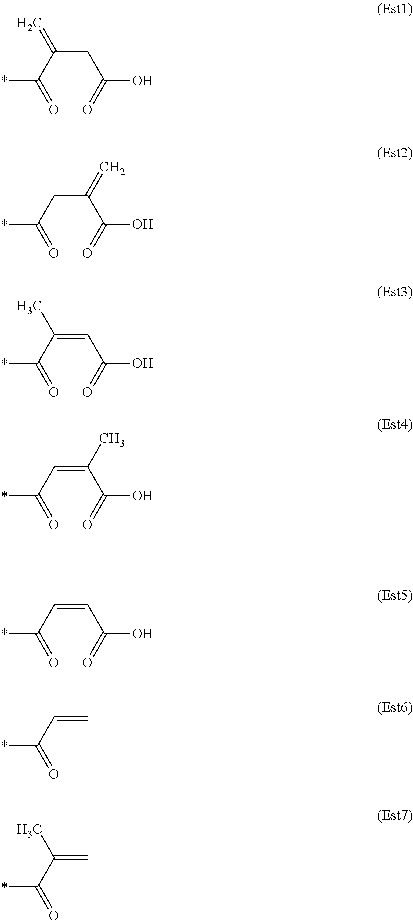

E is an ethane-1,2-diyl group,
P is a propane-1,2-diyl group or a propane-1,3-diyl group,
a, b and c are, independently of one another, a number from 0 to 100,
n is a number from 0 to 1000
x is a number 2 or 3,
v is a number from 0 to 200,
y is a number from 1 to 200 and
z is less than or equal to y.

The EO or PO or OE or OP fragments can be present in a block or statistically distributed in the monomer.

According to another preferred embodiment, the agent has as polymer (b) at least one polymer with silicone side chains and at least one anionic group formed by copolymerizing at least the monomers (i) to (iii),
(i) at least one monomer with a silicone side chain chosen from compounds of the Formulae (A1) and (A2),

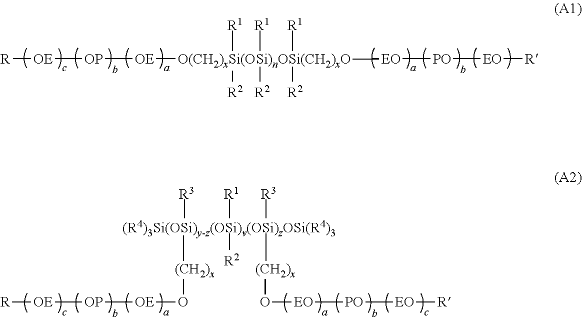

wherein
R¹ and R² are, independently of one another, a (C₁ to C₃₀) alkyl group, a (C₁ to C₂₀) alkyl group substituted with at least one halogen atom (e.g., —CCI₃, —CBr₃, —CF₃), a (C₃ to C₈) cycloalkyl group, a (C₆ to C₁₄) aryl group or (C₂ to C₂₀) alkenyl group,
R³ is a (C₁ to C₃₀) alkyl group, a (C₁ to C₂₀) alkyl group substituted with at least one halogen atom (e.g., —CCI₃, —CBr₃, —CF₃), a (C₃ to C₈) cycloalkyl group, a (C₆ to C₁₄) aryl group or (C₂ to C₂₀) alkenyl group,
R⁴ are each independently a (C₁ to C₃₀) alkyl group, a (C₆ to C₁₄) aryl group, a (C₂ to C₂₀) alkenyl group,
R and R′ at least one of R or R′ is an ester group of an olefinic unsaturated carboxylic acid according to one of the Formulae (Est1) to (Est7), wherein the other is one of these ester groups of Formulae (Est1) to (Est7) or is a hydrogen atom,

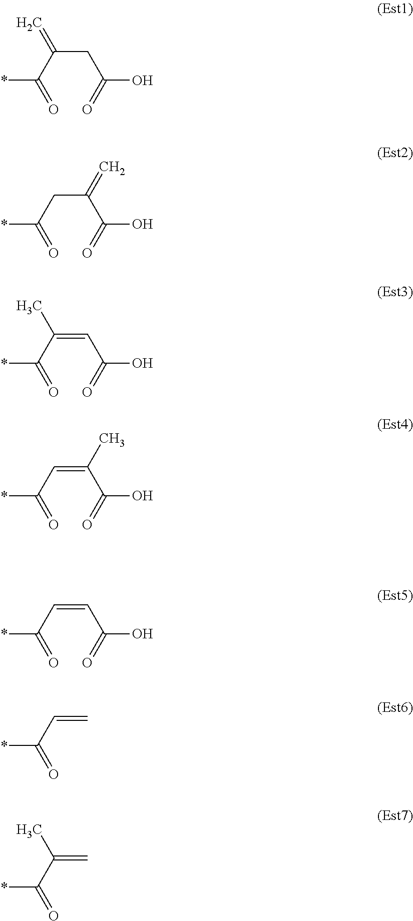

E is an ethane-1,2-diyl group,

P is a propane-1,2-diyl group or a propane-1,3-diyl group,

a, b and c are independently of one another, a number from 0 to 100,

n is a number from 0 to 1000

x is a number 2 or 3,

v is a number from 0 to 200,

y is a number from 1 to 200 and

z is less than or equal to y,
(ii) at least one additional anionic monomer, and
(iii) at least one additional monomer chosen from non-ionic monomers, cationic monomers, associative monomers or crosslinking monomers.

Preferably, x in Formulas (A1) or (A2) is the number 3.

Considering all preceding embodiments, it is particularly preferred when monomer (i) is chosen from at least one monomer according to Formulae (A1-1), (A1-2), (A2-1) and (A2-2)

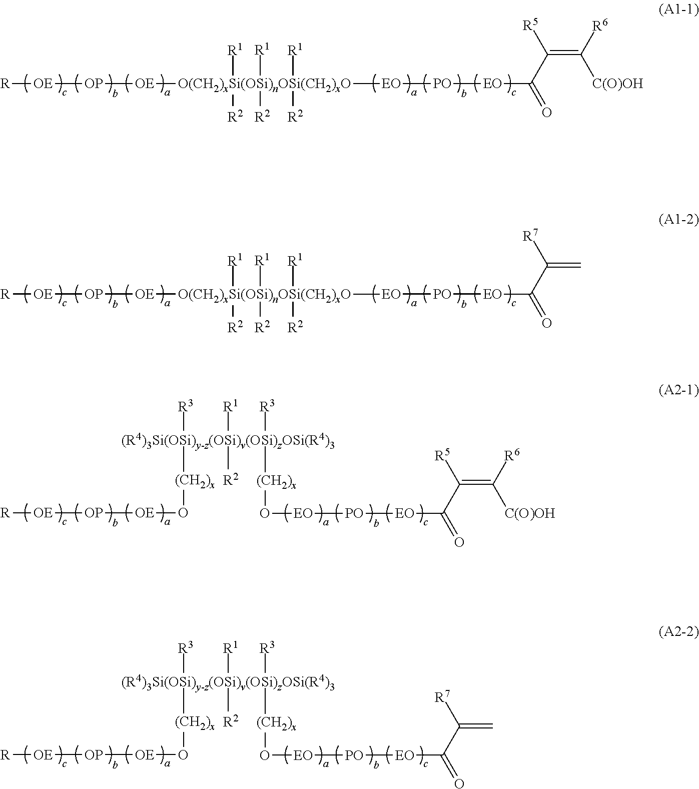

wherein
R¹, R², R³, R⁴, E, P, a, b, c, x, n, v, y and z are defined as in Formulae (A1) and (A2), and
R⁷ is a hydrogen atom or a methyl group,
wherein
one of the groups R⁵ or R⁶ is a methyl group and the other is a hydrogen atom, and R is a hydrogen atom or a group of Formulae (Est8) or (Est9),

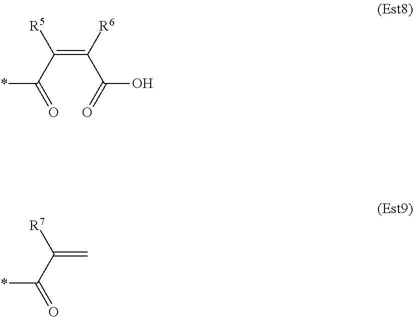

wherein R⁵, R⁶ and R⁷ are as described above.

Preferred suitable monomers are those wherein x is the number 3 according to Formulae (A1) or (A2) or (A1-1) or (A1-2) or (A2-1) or (A2-2).

According to all preceding embodiments, additional non-ionic monomers are preferably chosen from (C₁ to C₃₀) alkyl (meth)acrylates, cyclohexyl (meth)acrylates, 3,3,5-trimethylcyclohexyl (meth)acrylate, (C₁ to C₃₀) alkyl(meth)acrylamides, styrene, substituted styrenes (such as vinyltoluene derivatives; e.g., 2-methylstyrene, butylstyrene, isopropylstyrene, p-chlorostyrene), vinyl esters (e.g., vinyl acetate, vinyl butyrate, vinyl caprolate, vinyl pivalate, vinyl neodecanoate), unsaturated nitriles (e.g., methacrylonitrile, acrylonitrile), unsaturated silanes (such as trimethylvinylsilane, dimethylethylvinylsilane, allyldimethylphenylsilane, allyltrimethylsilane, 3-acrylamidopropyltrimethylsilane, 3-trimethylsilylpropyl methacrylate, (C₁ to C₆)-hydroxyalkyl(meth)acrylates (e.g., 2-hydroxyethyl methacrylate (HEMA), 2-hydroxyethyl acrylate (2-HEA), 3-hydroxypropyl acrylate, glycerin mono(meth)acryl ester, tris(hydroxymethyl)ethyl mono(meth)acrylate, pentaerythritol mono(meth)acrylate, N-hydroxymethyl(meth)acrylamide, 2-hydroxyethyl(meth)acrylamide, 3-hydroxypropyl(meth)acryl amide, (meth)acrylamide, t-octyl(meth)acrylamide, N-(2,3-dihydroxypropyl)acrylamide, t-butyl(meth)acrylamide, N-vinyl caprolactam, N-vinyl pyrrolidone, methacrylamidoethyl-N-ethylene urea (e.g., CH₂═C(CH₃)C(O)NHCH₂CH₂—N-ethylene urea), (C₁ to C₆) alkoxy substituted (meth)acrylates, (C₁ to C₆) alkoxy substituted (meth)acrylamides (such as methoxyethyl (meth)acrylate, 2-(2-ethoxyethyloxy)ethyl (meth)acrylate, allyl alcohol, glycerin monoallyl ether, 3-methyl-3-buten-1-ol.

The additional non-ionic monomers can be water-soluble. Moreover, the non-ionic monomers can be used as the lipophilic monomer.

According to the preceding embodiments, additional anionic monomers are preferably chosen from acidic, polymerizable, ethylenically unsaturated monomers, which preferably have a carboxylic group and/or sulfonic acid group and/or phosphoric acid group which affords an acid and also an anionic group after acid-base dissociation. These acidic groups are derived, for example, from mono or diacids, anhydrides of dicarboxylic acids, monoesters of dicarboxylic acids, or from the salts of the cited compounds.

Preferred additional anionic monomers are chosen from acrylic acid, methacrylic acid, itaconic acid, maleic acid, fumaric acid, crotonic acid, (C₁ to C₁₈) monoalkyl esters of maleic, fumaric, itaconic acid (such as methyl maleate, monoisopropyl maleate, butyl fumarate), maleic anhydride, itaconic anhydride, vinylsulfonic acid, 2-sulfoethyl methacrylate, p-styrene sulfonic acid, 2-acrylamido-2-methylpropane sulfonic acid (AMPS), allyloxybenzene sulfonic acid, vinylphosphonic acid, allylphosphonic acid, 3-acrylamidopropyl phosphonic acid, as well as the salts of the cited compounds.

Suitable salts of the cited additional anionic monomers include alkali metal salts (such as sodium, potassium and lithium salts) alkaline earth metal salts (such as calcium and magnesium salts) ammonium salts (NH₄⁺) and salts from the reaction with alkanolamines (such as with 2-amino-2-methyl-1-propanol, 2-aminoethanol, diethanolamine, triethanolamine, triethylamine).

It is also preferred to use at least two different additional anionic monomers for the synthesis of the anionic monomer with at least one silicone-containing side chain that is present in the agent.

Suitable additional cationic monomers are basic, polymerizable, ethylenically unsaturated monomers that preferably have at least one amino group. These basic amino groups are derived, for example, from mono, di or polyamino alkyl groups, or from nitrogen-containing heteroaromatic groups.

Suitable additional cationic monomers for the preceding embodiments are chosen from mono (C₁ to C₄) alkylamino (C₁ to C₆) alkyl (meth)acrylates, di-(C₁ to C₄) alkylamino(C₁ to C₈) alkyl (meth)acrylates, mono (C₁ to C₄) alkylamino (C₁ to C₈) alkyl (meth)acrylamides, di-(C₁ to C₄)-alkylamino(C₁ to C₈) alkyl(meth)acrylamides, nitrogen-containing heterocycle (meth)acrylamides, nitrogen-containing heterocycle (meth)acrylates, especially 2-(N,N-dimethylamino)ethyl (meth)acrylate (DMAEMA), 3-(N,N-dimethylamino)propyl (meth)acrylate, 4-(N,N-dimethylamino)butyl (meth)acrylate, (N,N-dimethylamino)-t-butyl (meth)acrylate, 2-(tert-butylamino)ethyl (meth)acrylate (TBAEMA), 2-(N,N-diethylamino)ethyl (meth)acrylate (DEAEMA), 3-(N,N-diethylamino)propyl (meth)acrylate, 2-(N,N-dimethylamino)neopentyl acrylate (DMANPA), 4-(N,N-diethylamino)butyl (meth)acrylate, 2-(N,N-dipropylamino)ethyl (meth)acrylate, 3-(N,N-dipropylamino)propyl (meth)acrylate, 4-(N,N-dipropylamino)butyl (meth)acrylate, 3-(N,N-dimethylamino)propyl (meth)acrylate, 2-(4-morpholinyl)ethyl (meth)acrylate, 2-(4-morpholinyl)ethyl acrylate, N′-(2-N,N-dimethylamino)ethyl (meth)acrylamide, 2-(N,N-dimethylamino)propyl (meth)acrylamide (DMAPMAm), N′-(3-N,N-dimethylamino)propyl (meth)acrylamide, N-(2-pyridyl)acrylamide, N-(2-imidazoyl) (meth)acrylamide, N-(4-morpholinyl) (meth)acrylamide, N-(4-morpholinyl)acrylamide, 2-vinylpyridine, 4-vinylpyridine, N-vinyl-2-methylimidazol, N-vinyl imidazole, 1-vinyl-3-methylimidazolium, N-vinyl oxazolidone, compounds according to Formula (Kat1)

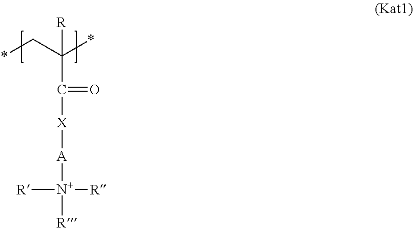

wherein
R is a hydrogen atom or a methyl group,
R′, R″ and R″ are, independently of one another, a (C₁ to C₂₀) alkyl group,
X is an oxygen atom or an NH group, and
A is an ethane-1,2-diyl group or a propane-1,3-diyl group,
as well as salts of all the cited compounds.

To compensate for the positive polymer charge of cationic monomer (ii), all possible physiologically acceptable anions can be used, such as chloride, bromide, hydrogen sulfate, methyl sulfate, ethyl sulfate, tetrafluoroborate, phosphate, hydrogen phosphate, dihydrogen phosphate or p-toluene sulfonate, or triflate. Suitable salts of the additional cationic monomers therefore include salts of mineral acids (such as hydrochloric acid or sulfuric acid), (C₁ to C₇) alkyl sulfates, phosphates, salts of organic acids (such as acetates, citrates, maleates, tartrates).

According to all previously cited embodiments, those additional associative monomers (hydrophobic monomers) are suitable for synthesis of the inventive polymers (b) which have an ethylenically unsaturated end group (1) for addition polymerization with other monomers of the monomer mixture, a polyoxyalkylene-containing middle group (2) for contribution to the hydrophilicity of the polymeric final product, and a hydrophobic end group for hydrophobic contribution of the polymeric final product.

The end group (1) is preferably derived from ethylenically unsaturated mono or dicarboxylic acids or their anhydrides, particularly preferably from mono or dicarboxylic acids containing 3 or 4 carbon atoms or their anhydrides. Furthermore, the end group (1) of the additional associative monomers can be derived from allyl ethers or vinyl ethers; non-ionic vinyl-substituted urethane monomers or vinyl-substituted urea derivatives.

The middle group (2) preferably contains a polyoxyalkylene segment of 5 to 250, particularly preferably 10 to 120, and quite particularly preferably 15 to 60 repeating (C₂ to C₆) alkylene oxide units.

Quite particularly preferred middle groups (2) include polyoxyethylene segments and/or polyoxypropylene segments and/or polyoxybutylene segments, comprising in all 5 to 150, preferably 10 to 100, particularly preferably 15 to 60 of said alkylene oxide units.

The hydrophobic end group (3) of the additional associative monomer preferably is a hydrocarbon group chosen from one of the following classes: linear (C₈ to C₄₀) alkyl groups, aryl-substituted (C₂ to C₄₀) alkyl groups, (C₂ to C₄₀) alkyl-substituted phenyl groups, branched (C₈ to C₄₀) alkyl groups, carbocyclic (C₈ to C₄₀) alkyl groups, esters with 8 to 80 carbon atoms as the complex ester. These complex esters refer to esterification products of a polyol with an saturated or unsaturated, long chain fatty acid with hydroxyl group(s). One skilled in the art understands “long chain” as being 10 to 30 carbon atoms. Exemplary suitable polyols are glycerin, sorbitol, pentaerythritol, trimethylol propane.

Exemplary suitable hydrophobic end groups (3) of the additional associative monomer are linear or branched, saturated or unsaturated hydrocarbon groups with 8 to 40 carbon atoms, such as capryl, isooctyl, decyl, lauryl, myristyl, cetyl, stearyl, isostearyl, arachidyl, behenyl, cerotyl, montanyl, melissyl, lacceryl, hydrogenated peanut oil, hydrogenated soybean oil, hydrogenated canola oil, hydrogenated tallow oil, and hydrogenated (C₁₀ to C₃₀) terpenes (such as hydrogenated geraniol, hydrogenated farnesol, and hydrogenated phytol).

Useful (C₂ to C₄₀) alkyl-substituted phenyl groups include octyiphenyl, nonylphenyl, decylphenyl, dodecylphenyl, hexadecylphenyl, octadecylphenyl, isooctylphenyl, sec-butylphenyl.

Useful carbocyclic (C₈ to C₄₀) alkyl groups include sterol derivatives (such as cholesterol, lanosterol, 7-dehydrocholesterol, phytosterol, stigmasterol, campesterol, ergosterol, mycosterol), cyclooctyl, cyclododecyl, adamantyl, decahydronaphthyl, pinene, hydrogenated retinol, camphor, isobornyl alcohol.

Useful aryl-substituted (C₂ to C₄₀) alkyl groups include styryl (e.g., 2-phenylethyl), distyryl (e.g., 2,4-diphenylbutyl), tristyryl (e.g., 2,4,6-triphenylhexyl), 4-phenylbutyl, 2-methyl-2-phenylethyl, tristyrylphenolyl.

Useful suitable esters with 8 to 80 carbon atoms as the complex esters include hydrogenated castor oil, 1,2-diacylglycerides (such as glycerin-1,2-distearate, glycerin-1,2-dipalmitate, glycerin-1,2-dimyristate, di-, tri- or polyesters of sugars (such as 3,4,6-tristearyl glucose, 2,3-dilauryl fructose), sorbitol esters.

Particularly preferred additional associative monomers include those according to Formula (Ass1)

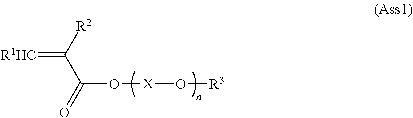

wherein
R¹ is a hydrogen atom, a carboxylic group, a (C₁ to C₂₂) alkoxycarbonyl group,
R² is a hydrogen atom or a methyl group,
R³ is a linear (C₈ to C₄₀) alkyl group, an aryl-substituted (C₂ to C₄₀) alkyl group, (C₂ to C₄₀)
alkyl-substituted phenyl groups, branched (C₈ to C₄₀) alkyl groups, carbocyclic (C₈ to C₄₀) alkyl groups, esters with 8 to 80 carbon atoms as the complex ester,
X is independently a C₂H₄ group, a C₃H₆ group or a C₄H₈ group,
n is a number from 5 to 250, preferably 5 to 100, more preferably 10 to 80, quite preferably 15 to 60.

Quite particularly preferred additional associative monomers of Formula (Ass1) are chosen from cetyl polyethoxylated methacrylate (CEM), stearyl polyethoxylated (meth)acrylate, arachidyl polyethoxylated (meth)acrylate, behenyl polyethoxylated methacrylate (BEM), lauryl polyethoxylated methacrylate (LEM), cerotyl polyethoxylated (meth)acrylate, montanyl polyethoxylated (meth)acrylate, melissyl polyethoxylated (meth)acrylate, lacceryl polyethoxylated (meth)acrylate, tristyrylphenol polyethoxylated methacrylate (TEM), hydrogenated castor oil polyethoxylated (meth)acrylate, canola polyethoxylated (meth)acrylate and cholesteryl polyethoxylated (meth)acrylate, wherein the number of polyethoxy units in monomer (ii) is 5 to 100, preferably 10 to 80, more preferably 15 to 60 ethylene oxide units.

The anionic polymer having at least one silicone-containing side chain is preferably crosslinked.

At least one additional crosslinking monomer is chosen from polyunsaturated aromatic monomers (such as divinylbenzene, divinylnaphthalene, trivinylbenzene), polyunsaturated alicyclic monomers (such as 1,2,4-trivinylcyclohexane), di-functional esters of phthalic acid (such as diallyl phthalate), polyunsaturated aliphatic monomers (such as dienes, trienes, tetraenes such as isoprene, 1,3-butadiene, 1,5-hexadiene, 1,5,9-decatriene, 1,9-decadiene, 1,5-heptadiene), polyalkenyl ethers (such as triallyl pentaerythritol, diallyl pentaerythritol, diallyl sucrose, octaallyl sucrose, trimethylolpropane diallyl ether), polyunsaturated esters of polyalcohols or polyacids (such as 1,6-hexane diol di(meth)acrylate, tetramethylene tri(meth)acrylate, allyl acrylate, diallyl itaconate, diallyl fumarate, diallyl maleate, trimethylolpropane tri(meth)acrylate, trimethylolpropane di(meth)acrylate, polyethylene glycol di(meth)acrylate), alkylene bisacrylamides (such as methylene bisacrylamide, propylene bisacrylamide) hydroxy and carboxy derivatives of methylene bisacrylamide (such as N,N′-bis-methylolmethylene bisacrylamide), polyethylene glycol di(meth)acrylates (such as ethylene glycol di(meth)acrylate, diethylene glycol di(meth)acrylate, triethylene glycol di(meth)acrylate), polyunsaturated silanes (such as dimethyldivinylsilane, methyltrivinylsilane, allyldimethylvinylsilane, diallyldimethylsilane, tetravinylsilane), N-methylolacrylamide; N-alkoxy(meth)acrylamides, wherein the alkoxy group is a (C₁ to C₁₈) alkoxy group, unsaturated hydrolyzable silanes (such as triethoxy vinylsilane, triisopropoxy vinylsilane, 3-triethoxy silylpropyl methacrylate), hydrolyzable silanes (such as ethyltriethoxysilane, ethyltrimethoxysilane), epoxy-substituted hydrolyzable silanes (such as 2-(3,4-epoxycyclohexyl)ethyltriethoxysilane, 3-glycidoxypropyltrimethoxysilane) polyisocyanates (such as 1,4-diisocyanatobutane, 1,6-diisocyanatohexane, 1,4-phenylenediisocyanate, 4,4′-oxybis(phenylisocyanate), unsaturated epoxides (such as glycidyl methacrylate, allyl glycidyl ether), polyepoxides (such as diglycidyl ether, 1,2,5,6-diepoxyhexane, ethylene glycol diglycidyl ether).

Particularly suitable polyunsaturated additional crosslinking monomers are derived from ethoxylated polyols such as diols, triols and diphenols, each ethoxylated with 2 to 100 mol ethylene oxide per mol hydroxyl groups and terminated with a polymerizable unsaturated group, such as vinyl ethers, allyl ethers, acrylate esters, methacrylate esters. Examples of such additional crosslinking monomers include ethoxylated Bisphenol A di(meth)acrylate, ethoxylated Bisphenol F di(meth)acrylate, ethoxylated trimethylolpropane tri(meth)acrylate, acrylate and methacrylate esters of polyols with at least two acrylate ester or methacrylate ester functionalities (such as for example trimethylolpropane triacrylate (TMPTA), trimethylolpropane ethoxylated (15) triacrylate (TMPEO15TA), trimethylolpropane dimethacrylate, triethylene glycol dimethacrylate (TEGDMA), Bisphenol A dimethacrylate ethoxylated with 30 mol ethylene oxide (EOBDMA).

Preferably, at least one anionic polymer with silicone side chains obtained by copolymerizing at least one of the monomers (i) to (v) is added to the agents
(i) at least one monomer having an olefinic unsaturated group and a silicone group, and
(ii) acrylic acid and/or methacrylic acid,
(iii) at least one additional non-ionic monomer chosen from (C₁ to C₄) alkyl esters of acrylic acid, (C₁ to C₄) alkyl esters of methacrylic acid,
(iv) optionally, at least one additional associative monomer, and
(v) optionally, at least one additional crosslinking monomer.

In this regard, all preferred embodiments of polymer (a) and all preferred embodiments of polymer (b) once again prove to be preferably suitable.

A preferred variant of polymer (b) is obtained by copolymerizing at least the monomers
(i) to (v)
(i) at least one monomer having an olefinic unsaturated group and a silicone group, and
(ii) acrylic acid and/or methacrylic acid,
(iii) at least one additional non-ionic monomer chosen from (C₁ to C₄) alkyl esters of acrylic acid, (C₁ to C₄) alkyl esters of methacrylic acid,
(iv) optionally, at least one additional associative monomer, and
(v) optionally at least one additional crosslinking monomer.

In this regard, all previously cited preferred embodiments regarding polymer (a) and all previously cited preferred embodiments regarding polymer (b) once again prove to be particularly preferably suitable.

A quite particularly preferred anionic polymer with silicone-containing side chains is marketed by Noveon under the trade name Fixate® Superhold (30 wt. % polymeric active substance, INCI name: Polyacrylate-2 Crosspolymer).

In a preferred embodiment, the agent further comprises, in addition to polymers (a) and (b), at least one film-forming and/or setting polymer.

Preferred properties of the film-forming polymers include film formation. Film-forming polymers refer to those polymers that, on drying, leave a continuous film on the skin, hair or nails. These types of film-former can be used in a wide variety of cosmetic products such as make up masks, make up, hair sets, hair sprays, hair gels, hair waxes, hair conditioners, shampoos or nail varnishes. Those polymers are particularly preferred which are sufficiently soluble in alcohol or water/alcohol mixtures, so that they are present in completely dissolved form in the agents. The film-forming polymers can be of synthetic or of natural origin.

According to the invention, film-forming polymers further refer to those polymers that, when used in concentrations of 0.01 to 20 wt. % in aqueous, alcoholic or aqueous alcoholic solution, are able to separate out a transparent polymer film on the hair.

Setting polymers contribute to the hold and/or creation of hair volume and hair body of the whole hairstyle. These polymers are also film-forming polymers and therefore are generally typical substances for styling hair treatment compositions such as hair sets, hair foams, hair waxes, hair sprays. Film formation can be in completely selected areas and bond only some fibers together.

The curl-retention test is frequently used as a test method for the setting action.

As polymers are often multifunctional (i.e., show a plurality of desired end-use effects), a large number of polymers are found in many of the groups divided according to the mode of action, also in the CTFA Handbook.

The agent preferably has at least one film-forming and/or setting polymer chosen from at least one polymer of the group of non-ionic polymers, cationic polymers, amphoteric polymers, zwitterionic polymers and anionic polymers.

The agent preferably contains additional film-forming and/or setting polymers in an amount of 0.01 wt. % to 20.0 wt. %, preferably 0.5 wt. % to 15.0 wt. %, and more preferably 2.0 wt. % to 10.0 wt. %, based on total weight of the agent. These quantitative data also apply for all subsequent preferred types of film-forming and/or setting polymers that can be used in the agents. Where subsequently different preferred quantities are specified, then the latter are to be again taken as the preferred quantities.

Those agents are particularly preferable that have, besides the previously defined amphiphilic, cationic polymers, at least one film-forming and/or setting polymer chosen from at least one polymer of the group of

non-ionic polymers based on ethylenically unsaturated monomers, especially from

homopolymers of N-vinyl pyrrolidone,

non-ionic copolymers of N-vinyl pyrrolidone,

homopolymers and non-ionic copolymers of N-vinyl caprolactam,

copolymers of (meth)acrylamide, and

polyvinyl alcohol, polyvinyl acetate,

chitosan and derivatives of chitosan,

cationic cellulose derivatives,

cationic copolymers of 3-(C₁ to C₆) alkyl-1-vinyl-imidazolinium,

homopolymers and copolymers comprising the structural unit of Formula (M−1)

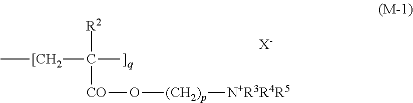

wherein R²=—H or —CH₃, R³, R⁴ and R⁵ are, independently of each other, chosen from (C₁ to C₄) alkyl, (C₁ to C₄) alkenyl or (C₂ to C₄) hydroxyalkyl groups, p=1, 2, 3 or 4, q is a natural number and X⁻ is a physiologically acceptable organic or inorganic anion,

anionic polymers having carboxylate and/or sulfonate groups, and

anionic polyurethanes.

Preferred non-ionic polymers, based on ethylenically unsaturated monomers suitable as the film-forming and/or setting polymers are those non-ionic polymers having at least one of the following structural units

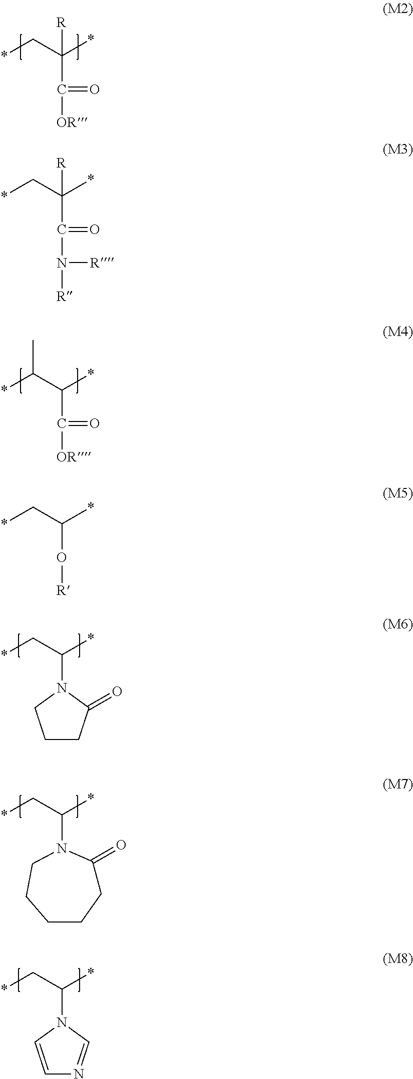

wherein
R is a hydrogen atom or a methyl group,
R′ is a hydrogen atom or a (C₁ to C₄) acyl group,
R″ and R″″ are, independently of one another, a (C₁ to C₇) alkyl group or a hydrogen atom, and
R′″ is a linear or branched (C₁ to C₄) alkyl group or a (C₂ to C₄) hydroxyalkyl group.

Suitable, non-ionic film-forming and/or non-ionic hair setting polymers are homopolymers or copolymers based on at least one of the following monomers: vinyl pyrrolidone, vinyl caprolactam, vinyl esters such as vinyl acetate, vinyl alcohol, acrylamide, methacrylamide, alkyl and dialkyl acrylamides, alkyl and dialkyl methacrylamides, alkyl acrylates, alkyl methacrylates, wherein each of the alkyl groups of these monomers are chosen from (C₁ to C₃) alkyl groups.

For agents according to the invention, particularly suitable non-ionic polymers based on ethylenically unsaturated monomers contain at least one of the following structural units

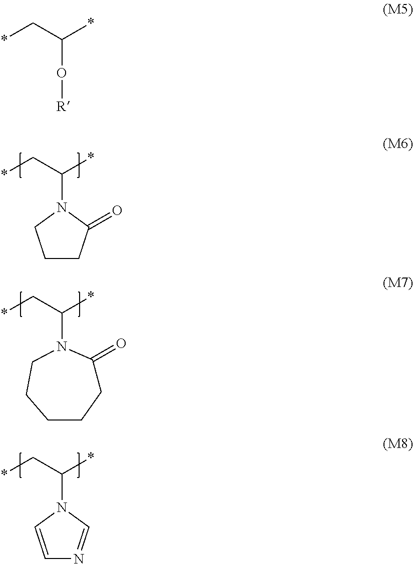

wherein R′ is a hydrogen atom or a (C₁ to C₃₀) acyl group, particularly a hydrogen atom or an acetyl group.

Homopolymers of vinyl caprolactam or of vinyl pyrrolidone (such as Luviskol® K 90 or Luviskol® K 85 from BASF SE), copolymers of vinyl pyrrolidone and vinyl acetate (such as are marketed under the trade names Luviskol® VA 37, Luviskol® VA 55, Luviskol® VA 64 and Luviskol® VA 73 by BASF SE), terpolymers of vinyl pyrrolidone, vinyl acetate and vinyl propionate, polyacrylamides (such as Akypomine® P 191 from CHEM-Y), polyvinyl alcohols (that are marketed for example under the trade names Elvanol® by Du Pont or Vinol® 523/540 by Air Products), terpolymers of vinyl pyrrolidone, methacrylamide and vinyl imidazole (such as Luviset® Clear from BASF SE) are particularly suitable.

In addition to non-ionic polymers based on ethylenically unsaturated monomers, non-ionic cellulose derivatives are also suitable film-forming and/or setting polymers. They are preferably chosen from methyl cellulose, especially from cellulose ethers such as hydroxypropyl cellulose (e.g., hydroxypropyl cellulose with a molecular weight of 30,000 to 50,000 g/mol, marketed, for example, under the trade name Nisso SI® by Lehmann & Voss, Hamburg), and hydroxyethyl celluloses such as are marketed under the trade names Culminal® and Benecel® (AQUALON) and Natrosol® types (Hercules).

Cationic polymers refer to polymers that, in their main chain and/or side chain, have groups that can be “temporarily” or “permanently” cationic. “Permanently cationic” refers to those polymers that, independently of the pH of the medium, have a cationic group. These are generally polymers having a quaternary nitrogen atom, for example, in the form of an ammonium group. Preferred cationic groups are quaternary ammonium groups. In particular, those polymers wherein the quaternary ammonium groups are bonded through a C₁₋₄ hydrocarbon group to a polymer backbone formed from acrylic acid, methacrylic acid or their derivatives have proved to be particularly suitable.

An preferred suitable cationic film-forming and/or cationic setting polymer is at least one cationic film-forming and/or cationic setting polymer having at least one structural element of Formula (M9) and additionally at least one structural element of Formula (M10)

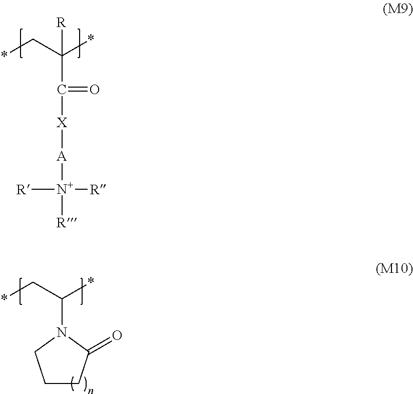

wherein
R is a hydrogen atom or a methyl group,
R′, R″ and R′″ are, independently of one another, a (C₁ to C₆) alkyl group,
X is an oxygen atom or an NH group,
A is an ethane-1,2-diyl group or a propane-1,3-diyl group, and
n is 1 or 3.

To compensate for the positive polymer charge, all possible physiologically acceptable anions can be used, such as chloride, bromide, hydrogen sulfate, methyl sulfate, ethyl sulfate, tetrafluoroborate, phosphate, hydrogen phosphate, dihydrogen phosphate or p-toluene sulfonate, or triflate. Exemplary compounds of this type are
copolymers of dimethylaminoethyl methacrylate quaternized with diethyl sulfate, with vinyl pyrrolidone having the INCI name Polyquatemium-11 under the trade names Gafquat® 440, Gafquat® 734, Gafquat® 755 (each from ISP) and Luviquat PQ 11 PN (BASF SE).

Furthermore, cationic film-forming and/or cationic setting polymers are preferably chosen from cationic, quaternized cellulose derivatives.

Moreover, cationic, quaternized cellulose derivatives are preferred suitable film-forming and/or setting polymers.

Those cationic, quaternized celluloses having more than one permanent cationic charge in a side chain have proven to be particularly advantageous. Among these cationic celluloses, those cationic celluloses with the INCI name Polyquaternium-4 are particularly suitable, marketed, for example, by the National Starch company under the trade names Celquat® H 100, Celquat® L 200.

In the context of the invention, those cationic film-forming and/or cationic setting copolymers having at least one structural element of Formula (M11) additionally serve as particularly preferred usable cationic polymers

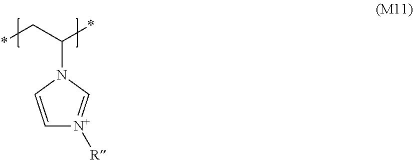

wherein R″ is a (C₁ to C₄) alkyl group, particularly a methyl group, and additionally having at least one further cationic and/or non-ionic structural element.

To compensate for the positive polymer charge, all possible physiologically acceptable anions can be used, such as chloride, bromide, hydrogen sulfate, methyl sulfate, ethyl sulfate, tetrafluoroborate, phosphate, hydrogen phosphate, dihydrogen phosphate or p-toluene sulfonate, or triflate.

Among these are vinyl pyrrolidone/1-vinyl-3-methyl-1H-imidazolium chloride copolymers (such as that having the INCI name Polyquaternium-16 sold under the trade names Luviquat® Style, Luviquat® FC 370, Luviquat® FC 550, Luviquat® FC 905 and Luviquat® HM 552 (BASF SE)), vinyl pyrrolidone/1-vinyl-3-methyl-1H-imidazolium methylsulfate copolymers (such as that having the INCI name Polyquatemium-44 sold under the trade name Luviquat® Care (BASF SE)), vinyl pyrrolidone/vinyl caprolactam/1-vinyl-3-methyl-1H-imidazolium terpolymer (such as that having the INCI name Polyquatemium-46 sold under the trade names Luviquat® Care or Luviquat® Hold (BASF SE)), vinyl pyrrolidone/methacrylamide/vinyl imidazole/1-vinyl-3-methyl-1H-imidazolium methyl sulfate copolymer (such as that having the INCI name Polyquatemium-68 sold under the trade name Luviquat® Supreme (BASF SE)).

Further preferred cationic polymers that can be used in the agents are the “temporarily cationic” polymers. These polymers usually have an amino group that is present at specific pH values as a quaternary ammonium group and is thus cationic.

These polymers include, for example, chitosan. In the context of the present invention, chitosan and/or chitosan derivatives are considered as quite particularly preferred suitable film-forming and/or setting polymers.

Chitosans are biopolymers and are considered to be hydrocolloids. From a chemical point of view, they are partially deacetylated chitins of different molecular weight.

Chitosan is manufactured from chitin, preferably from the remains of crustacean shells, which are available in large quantities as a cheap raw material. For this, the chitin is firstly deproteinated by adding bases, demineralized by adding mineral acids and finally deacetylated by adding strong bases, wherein the molecular weights can vary over a wide spectrum. Those types are preferably employed having an average molecular weight of 800,000 to 1,200,000 Dalton, a viscosity according to Brookfield (1 wt. % conc. in glycolic acid) of 5000 mPas or less, a deacetylation degree in the range of 80 to 88% and an ash content of 0.3 wt. % or less.

In addition to chitosans as typical cationic biopolymers, cationically derivatized chitosans can also be considered (such as quaternized products) or alkoxylated chitosans.

Preferred agents contain neutralization products of chitosan neutralized with at least one acid chosen from lactic acid, pyrrolidone carboxylic acid, nicotinic acid, hydroxy-iso-butyric acid, hydroxy-iso-valeric acid, or contain mixtures of these neutralization products as the chitosan derivative(s).

Exemplary suitable chitosan (derivatives) are freely available on the market under the trade names Hydagen® CMF (1 wt. % active substance in aqueous solution with 0.4 wt. % glycolic acid, molecular weight 500,000 to 5,000,000 g/mol Cognis), Hydagen® HCMF (chitosan (80% deacetylated), molecular weight 50,000 to 1,000,000 g/mol, Cognis), Kytamer® PC (80 wt. % active substance of chitosan pyrrolidone carboxylate (INCI name: Chitosan PCA), Amerchol) and Chitolam® NB/101.

Agents according to the invention preferably contain chitosan or its derivatives in an amount of 0.01 wt. % to 20.0 wt. %, preferably 0.01 wt. % to 10.0 wt. %, more preferably 0.1 wt. % to 1 wt. %, based on total weight of the agent.

Preferred temporarily cationic polymers are likewise those having at least one structural unit according to Formulae (M1-1) to (M1-8)

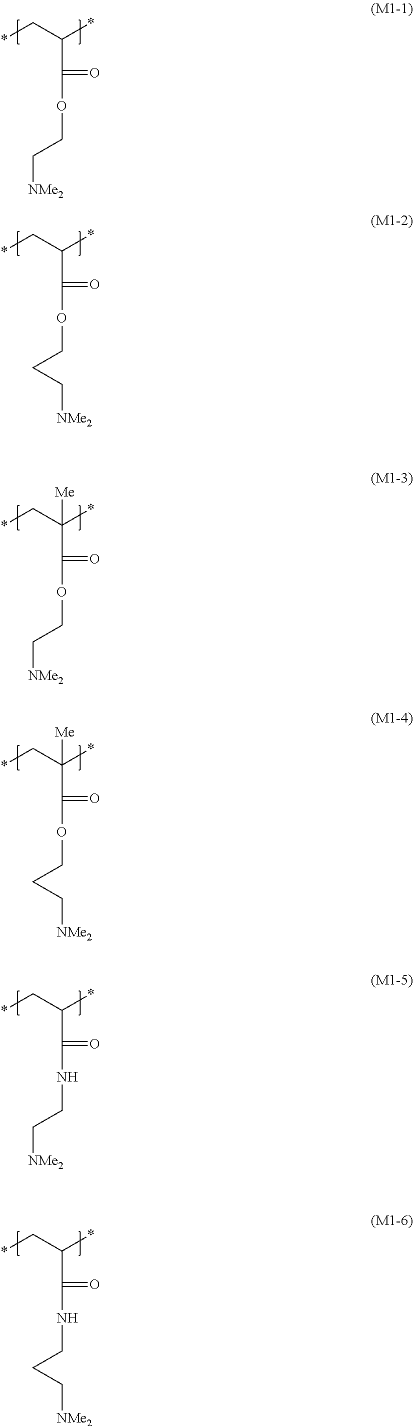

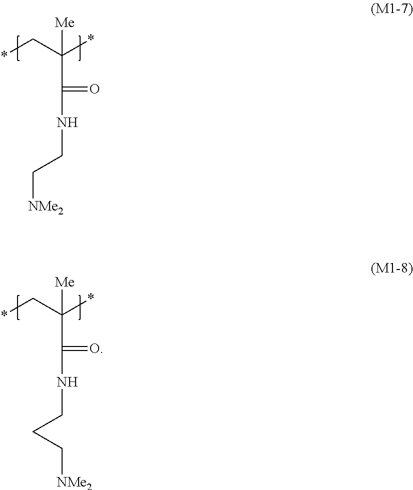

In this regard, those copolymers are preferred that have at least one structural unit of Formulae (M1-1) to (M1-8) and additionally at least one structural unit of the Formula (M10),

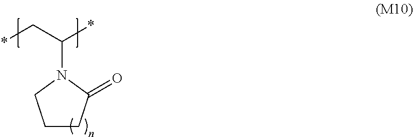

wherein n is 1 or 3.

A preferred list of polymers for selection include
vinyl caprolactam/vinyl pyrrolidone/dimethylaminoethyl methacrylate copolymer (for example, INCI name: Vinyl Caprolactam/PVP/Di-methylaminoethyl Methacrylate Copolymer under the trade name Gaffix® VC 713 (ISP)),
vinyl pyrrolidone/vinyl caprolactam/dimethylaminopropylmethacrylamide copolymer (for example, INCI name: VP/Vinyl Caprolactam/DMAPA Acrylates Copolymer under the trade name Aquaflex® SF-40 (ISP)),
vinyl caprolactam/vinyl pyrrolidone/dimethylaminoethyl methacrylate copolymer (for example, as a 35-39% solids in ethanol commercially available as Advantage LC E with the INCI name: Vinyl Caprolactam/VP/Dimethylaminoethyl Methacrylate Copolymer, Alcohol, Lauryl Pyrrolidone (ISP)), and
vinyl pyrrolidone/dimethylaminopropylmethacrylamide copolymer (for example INCI name: VP/DMAPA Acrylates Copolymer under the trade name Styleze® CC-10 (ISP)).

The agents can also have at least one amphoteric polymer as the film-forming and/or setting polymer. The term amphopolymers includes not only those polymers whose molecule has both free amino groups and free —COOH or SO₃H groups and which are capable of forming inner salts, but also zwitterionic polymers whose molecule has quaternary ammonium groups and —COO— or —SO₃— groups, and polymers having —COOH or SO₃H groups and quaternary ammonium groups.

An example of an amphopolymer which may be used according to the invention is the acrylic resin available under the designation Amphomer®, which is a copolymer of tert-butylaminoethyl methacrylate, N-(1,1,3,3-tetramethylbutyl)acrylamide, and two or more monomers from the group of acrylic acid, methacrylic acid and their simple esters.

The latter, in addition to a cationic or positively charged group, have at least one negatively charged group in the molecule and are also called zwitterionic polymers. The agents preferably included amphoteric polymers in amounts of 0.01 to 20 wt. %, more preferably 0.05 to 10 wt. %, based on total weight of the agent. Quantities of 0.1 to 5 wt. % are particularly preferred.

At least one anionic film-forming and/or anionic setting polymer can also be used as the film-forming and/or setting polymers.

Anionic polymers are polymers having carboxylate and/or sulfonate groups. Examples of anionic monomers from which such polymers can be made are acrylic acid, methacrylic acid, crotonic acid, maleic anhydride and 2-acrylamido-2-methylpropane sulfonic acid. Here, the acidic groups may be fully or partially present as sodium, potassium, ammonium, mono- or triethanolammonium salts.

Within this embodiment, preferably copolymers of at least one anionic monomer and at least one non-ionic monomer are used. Regarding the anionic monomers, reference is made to the abovementioned substances. Preferred non-ionic monomers are acrylamide, methacrylamide, acrylic acid esters, methacrylic acid esters, vinyl pyrrolidone, vinyl ethers and vinyl esters.

Preferred anionic copolymers are acrylic acid-acrylamide copolymers, particularly polyacrylamide copolymers with monomers having sulfonic acid groups. A particularly preferred anionic copolymer is 70 to 55 mole % acrylamide and 30 to 45 mole % 2-acrylamido-2-methylpropane sulfonic acid, wherein the sulfonic acid group can be fully or partially present as the sodium, potassium, ammonium, mono- or triethanolammonium salt. This copolymer can also be crosslinked, with preferred crosslinking agents including polyolefinically unsaturated compounds such as tetraallyloxyethane, allyl sucrose, allyl pentaerythritol and methylene bisacrylamide. Such a polymer is present in the commercial product Sepigel®305 from the SEPPIC company. Use of this compound, which has a mixture of hydrocarbons (C₁₃-C₁₄ isoparaffins) and a non-ionic emulsifier (Laureth-7) in addition to the polymer components, has proved to be particularly advantageous.

Sodium acryloyl dimethyl taurate copolymers commercialized as a compound with isohexadecane and polysorbate 80 under the trade name Simulgel®600 have also proved to be particularly effective.

Likewise preferred anionic homopolymers include uncrosslinked and crosslinked polyacrylic acids. Here, the preferred crosslinking agents can be allyl ethers of pentaerythritol, sucrose and propylene. Such compounds are commercially available under the trade name Carbopol®, for example.

Further preferred employable anionic polymers are chosen from:
copolymers of vinyl acetate and crotonic acid (marketed, for example, as the product Aristoflex® A 60 with the INCI name VA/Crotonates Copolymer by CIBA in a 60 wt. % conc. dispersion in isopropanol-water),
copolymers of ethyl acrylate and methacrylic acid (marketed, for example, under the trade name Luviflex® Soft with an acid number of 84 to 105 with the INCI name Acrylates Copolymer in a ca. 20 to 30 wt. % conc. dispersion in water by BASF SE), and
Polyurethanes containing at least one carboxylic group (such as a copolymer of isophthalic acid, adipic acid, 1,6-hexane diol, neopentyl glycol and isophorone diisocyanate, marketed under the trade name Luviset® PUR with the INCI name Polyurethane-1 by BASF SE).

When particularly strong acting thickening anionic polymers are used, then care should be taken that the previously cited preferred viscosity criterion of the agent is adhered to.

Copolymers of maleic anhydride and methyl vinyl ether, especially those with crosslinks are also color-conserving polymers. A maleic acid-methyl vinyl ether copolymer crosslinked with 1,9-decadiene is commercially available under the name Stabileze® QM.

In order to intensify the effect according to the invention, the agents preferably also have at least one surfactant, wherein non-ionic, anionic, cationic, ampholytic surfactants are suitable. Ampholytic or amphoteric surfactants includes zwitterionic surfactants and ampholytes. According to the invention, the surfactants can already have an emulsifying action.

The agent preferably contain additional surfactants in an amount of 0.01 wt. % to 5 wt. %, preferably 0.05 wt. % to 0.5 wt. %, based on total weight of the agent.

It is particularly preferable when the agents have at least one non-ionic surfactant.

Non-ionic surfactants include, for example, a polyol group, a polyalkylene glycol ether group, or a combination of polyol ether groups and polyglycol ether groups as the hydrophilic group.

Examples of compounds of this type include
addition products of 2 to 100 moles ethylene oxide and/or 1 to 5 moles propylene oxide to linear and branched fatty alcohols containing 8 to 30 carbon atoms, to fatty acids containing 8 to 30 carbon atoms and to alkyl phenols containing 8 to 15 carbon atoms in the alkyl group,
methyl or C₂-C₆ alkyl group end blocked addition products of 2 to 50 moles ethylene oxide and/or 1 to 5 moles propylene oxide to linear and branched fatty alcohols with 8 to 30 carbon atoms, to fatty acids with 8 to 30 carbon atoms and to alkyl phenols with 8 to 15 carbon atoms in the alkyl group, such as, for example, the commercially available types Dehydrol® LS, Dehydrol® LT (Cognis),
C₁₂-C₃₀ fatty acid mono- and diesters of addition products of 1 to 30 moles ethylene oxide to glycerin,
addition products of 5 to 60 moles ethylene oxide on castor oil and hydrogenated castor oil,
polyol esters of fatty acids, such as the commercial product Hydagen® HSP (Cognis) or Sovermol types (Cognis),
alkoxylated triglycerides,
alkoxylated alkyl esters of fatty acids of Formula (E4-I),

RⁱCO−(OCH₂CHR²)_{w}OR³ (E4-I)
wherein R¹CO is a linear or branched, saturated and/or unsaturated acyl group with 6 to 22 carbon atoms, R² is hydrogen or methyl, R³ is linear or branched alkyl groups with 1 to 4 carbon atoms, and w is a number from 1 to 20,
amine oxides,
mixed hydroxy ethers, such as described in DE-OS 1 973 8866,
sorbitol esters of fatty acids and addition products of ethylene oxide to sorbitol esters of fatty acids such as the polysorbates,
sugar esters of fatty acids and addition products of ethylene oxide to sugar esters of fatty acids,
addition products of ethylene oxide to fatty acid alkanolamides and fatty amines,
sugar surfactants of the type alkyl and alkenyl oligoglycosides according to Formula (E4-II),

R⁴O−[G]ₚ (E4-II)
wherein R⁴ is an alkyl or alkenyl group containing 4 to 22 carbon atoms, G is a sugar group containing 5 or 6 carbon atoms, and p is a number from 1 to 10.

Alkylene oxide addition products to saturated, linear fatty alcohols and fatty acids, each with 2 to 100 moles ethylene oxide per mole fatty alcohol or fatty acid, are quite particularly preferred non-ionic surfactants. Similarly, preparations with excellent properties are obtained when they have C₁₂-C₃₀ fatty acid mono- and diesters of addition products of 1 to 30 moles ethylene oxide to glycerin and/or addition products of 5 to 60 moles ethylene oxide to castor oil and hydrogenated castor oil as the non-ionic surfactants.

Suitable anionic surfactants include all anionic surface-active materials suitable for use on the human body. They have a water solubilizing anionic group such as e.g. a carboxylate, sulfate, sulfonate or phosphate group, and a lipophilic alkyl group containing about 8 to 30 carbon atoms. In addition, the molecule can have glycol or polyglycol ether groups, ester, ether and amide groups, as well as hydroxyl groups. Examples of suitable anionic surfactants, each in the form of the sodium, potassium and ammonium as well as the mono, di and trialkanolammonium salts containing 2 to 4 carbon atoms in the alkanol group, are
linear and branched fatty acids with 8 to 30 carbon atoms (soaps),
ether carboxylic acids of the formula R—O—(CH₂—CH₂O)ₓ—CH₂—COOH, wherein R is a linear alkyl group with 8 to 30 carbon atoms and x=0 or 1 to 16,
acyl sarcosides with 8 to 24 carbon atoms in the acyl group,
acyl taurides with 8 to 24 carbon atoms in the acyl group,
acyl isethionates with 8 to 24 carbon atoms in the acyl group,
mono- and dialkyl esters of sulfosuccinic acid with 8 to 24 carbon atoms in the alkyl group and mono-alkyl polyoxyethyl esters of sulfosuccinic acid with 8 to 24 carbon atoms in the alkyl group and 1 to 6 oxyethylene groups,
linear alkane sulfonates containing 8 to 24 carbon atoms,
linear alpha-olefin sulfonates containing 8 to 24 carbon atoms,
alpha-sulfo fatty acid methyl esters of fatty acids containing 8 to 30 carbon atoms,
alkyl sulfates and alkyl polyglycol ether sulfates of the Formula R—O(CH₂-CH₂O)ₓ—OSO₃H, wherein R is preferably a linear alkyl group containing 8 to 30 carbon atoms and x=0 or 1 to 12,
mixtures of surface active hydroxyl sulfonates, sulfated hydroxyalkyl polyethylene glycol ethers and/or hydroxyalkylene propylene glycol ethers,
sulfonates of unsaturated fatty acids with 8 to 24 carbon atoms and 1 to 6 double bonds,
esters of tartaric acid and citric acid with alcohols, representing the addition products of about 2-15 molecules of ethylene oxide and/or propylene oxide on fatty alcohols containing 8 to 22 carbon atoms,
sulfated fatty acid alkylene glycol esters of Formula (E1-II)

R⁷CO(AIkO)ₙSO₃M (E1-II)
wherein R⁷CO— is a linear or branched, aliphatic, saturated and/or unsaturated acyl group containing 6 to 22 carbon atoms, Alk is CH₂CH₂, CHCH₃CH₂ and/or CH₂CHCH₃, n is a number from 0.5 to 5, and M is a cation, as described in DE-OS 197 36 906,
amido ether carboxylic acids, and
condensation products of C₈-C₃₀ fatty alcohols with protein hydrolyzates and/or amino acids and their derivatives, which are known to the person skilled in the art as albumin fatty acid condensates, such as for example the Lamepon® types, Gluadin® types, Hostapon® KCG or the Amisoft® types.

Preferred anionic surfactants are alkyl sulfates and ether carboxylic acids with 10 to 18 C atoms in the alkyl group and up to 12 glycol ether groups in the molecule, sulfosuccinic acid mono and dialkyl esters with 8 to 18 C atoms in the alkyl group and sulfosuccinic acid mono-alkyl polyoxyethyl esters with 8 to 18 C atoms in the alkyl group and 1 to 6 oxyethylene groups, monoglycerin disulfates, alkyl- and alkenyl ether phosphates, as well as albumin fatty acid condensates.

Zwitterionic surfactants are those surface-active compounds having at least one quaternary ammonium group and at least one —COO⁽⁻⁾ or —SO₃⁽⁻⁾ group in the molecule. Particularly preferred suitable zwitterionic surfactants are betaines such as N-alkyl-N,N-dimethylammonium glycinates, for example, cocoalkyl-dimethylammonium glycinate, N-acyl-aminopropyl-N,N-dimethylammonium glycinate, for example, coco-acylaminopropyl-dimethylammonium glycinate, and 2-alkyl-3-carboxymethyl-3-hydroxyethyl-imidazolines, each with 8 to 18 carbon atoms in the alkyl or acyl group, as well as cocoacyl-aminoethylhydroxyethylcarboxymethyl glycinate. A preferred zwitterionic surfactant is the fatty acid amide derivative known under the INCI name Cocamidopropyl Betaine.

Ampholytes include such surface-active compounds that, apart from a C₈₋₂₄ alkyl or acyl group, have at least one free amino group and at least one —COOH or —SO₃H group in the molecule and are able to form internal salts. Examples of suitable ampholytes are N-alkylglycines, N-alkyl propionic acids, N-alkylamino butyric acids, N-alkylimino dipropionic acids, N-hydroxyethyl-N-alkylamidopropylglycines, N-alkyltaurines, N-alkylsarcosines, 2-alkylamino propionic acids and alkylamino acetic acids, each with about 8 to 24 carbon atoms in the alkyl group. Particularly preferred ampholytes are N-cocoalkylamino propionate, cocoacylaminoethylamino propionate and C₁₂₋₁₈ acyl sarcosine.

Agents according to the invention contain ingredients or active substances in a cosmetically acceptable carrier. Preferred cosmetically acceptable carriers are aqueous, alcoholic or aqueous alcoholic media preferably having at least 10 wt. % water, based on total composition. In particular, lower alcohols containing 1 to 4 carbon atoms, such as ethanol and isopropanol, typically used for cosmetic purposes, can be used as alcohols.

Organic solvents or mixture of solvents with a boiling point of 400° C. or less can be used as additional co-solvents in an amount of 0.1 to 15 wt. %, preferably 1 to 10 wt. %, based on total agent. Particularly suitable additional co-solvents are unbranched or branched hydrocarbons such as pentane, hexane, isopentane, and cyclic hydrocarbons such as cyclopentane and cyclohexane. Additional, particularly preferred water-soluble solvents are glycerin, ethylene glycol and propylene glycol in an amount of up to 30 wt. %, based on total agent.

The addition of glycerin and/or propylene glycol and/or polyethylene glycol and/or polypropylene glycol increases the flexibility of the polymer film formed when the agent is used. Consequently, if a more flexible hold is desired, then the agents preferably have 0.01 to 30 wt % glycerin and/or propylene glycol and/or polyethylene glycol and/or polypropylene glycol, based on total agent.

The agents preferably have a pH of 2 to 11. The pH range is particularly preferably from 2 to 8. Here, pH data refers to pH at 25° C. unless otherwise stated.

The agents according to the invention can also include auxiliaries and additives typically incorporated into styling agents. In particular, care products are suitable auxiliaries and additives. According to the invention, at least one silicone oil and/or at least one silicone gum is preferably used as the care substance.

Suitable silicone oils or silicone gums according to the invention include dialkyl and alkylarylsiloxanes, such as dimethylpolysiloxane and methylphenylpolysiloxane, as well as their alkoxylated, quaternized or also anionic derivatives. Cyclic and linear polydialkylsiloxanes, their alkoxylated and/or aminated derivatives, dihydroxypolydimethylsiloxanes and polyphenylalkylsiloxanes are preferred.

Silicone oils afford a wide variety of effects. Thus, for example, they simultaneously influence dry and wet combability, feel of dry and wet hair, as well as gloss. The term “silicone oils” refers to organosilicon compounds with a plurality of structures. In the first instance they include Dimethiconols.

The following commercial products are examples of such products: Botanisil NU-150M (Botanigenics), Dow Corning 1-1254 Fluid, Dow Corning 2-9023 Fluid, Dow Corning 2-9026 Fluid, Ultrapure Dimethiconol (Ultra Chemical), Unisil SF-R (Universal Preserve), X-21-5619 (Shin-Etsu Chemical Co.), Abil OSW 5 (Degussa Care Specialties), ACC DL-9430 Emulsion (Taylor Chemical Company), AEC Dimethiconol & Sodium Dodecylbenzene sulfonate (A & E Connock (Perfumery & Cosmetics) Ltd.), B C Dimethiconol Emulsion 95 (Basildon Chemical Company, Ltd.), Cosmetic Fluid 1401, Cosmetic Fluid 1403, Cosmetic Fluid 1501, Cosmetic Fluid 1401 DC (all from Chemsil Silicones, Inc.), Dow Corning 1401 Fluid, Dow Corning 1403 Fluid, Dow Corning 1501 Fluid, Dow Corning 1784 HVF Emulsion, Dow Corning 9546 Silicone Elastomer Blend (all from Dow Corning Corporation), Dub Gel S11400 (Stearinerie Dubois Fils), HVM 4852 Emulsion (Crompton Corporation), Jeesilc 6056 (Jeen International Corporation), Lubrasil, Lubrasil DS (both from Guardian Laboratories), Nonychosine E, Nonychosine V (both from Exsymol), SanSurf Petrolatum-25, Satin Finish (both from Collaborative Laboratories, Inc.), Silatex-D30 (Cosmetic Ingredient Resources), Silsoft 148, Silsoft E-50, Silsoft E-623 (all from Crompton Corporation), SM555, SM2725, SM2765, SM2785 (all from GE Silicones), Taylor T-SiI CD-1, Taylor TME-4050E (all from Taylor Chemical Company), TH V 148 (Crompton Corporation), Tixogel CYD-1429 (Sud-Chemie Performance Additives), Wacker-Belsil CM 1000, Wacker-Belsil CM 3092, Wacker-Belsil CM 5040, Wacker-Belsil DM 3096, Wacker-Belsil DM 3112 VP, Wacker-Belsil DM 8005 VP, Wacker-Belsil DM 60081 VP (all from Wacker-Chemie GmbH).

Dimethicones form the second group of useful silicones according to the invention. They can be linear, branched, cyclic, or cyclic and branched. Dimethicone copolyols form a further group of suitable silicones. Suitable Dimethicone copolyols are commercially available and marketed, for example, by Dow Corning under the trade name Dow Corning® 5330 Fluid.

Naturally, the Dimethiconols, Dimethicones and/or Dimethicone copolymers can already be present as an emulsion. When the Dimethiconols, Dimethicones and/or Dimethicone copolyols are used as an emulsion, then droplet size of the emulsified particles can range from 0.01 to 10,000 μm, preferably 0.01 to 100 μm, more preferably 0.01 to 20 μm and quite preferably 0.01 to 10 μm. Particle size is determined according to the light scattering method.

Further suitable silicones are amino-functional silicones, especially those silicones compiled under the INCI name Amodimethicone. Consequently, it is preferred when the agents additionally contain at least one amino-functional silicone. These are silicones having at least one, optionally substituted, amino group. These silicones are designated as Amodimethicones according to the INCI nomenclature and are available, for example, in the form of an emulsion as the commercial product Dow Corning® 939 or as the commercial product Dow Corning® 949 in a mixture with a cationic and a non-ionic surfactant.

Preferably, those amino functional silicones are used which have an amine number of 0.25 meq/g or greater, preferably 0.3 meq/g or greater, and more preferably 0.4 meq/g or greater. The amine number is the milli-equivalents of amine per gram of the amino functional silicone. It can be measured by titration and can also be reported with the unit mg KOH/g.

The agents preferably include silicones in amounts of 0.01 wt. % to 15 wt. %, more preferably 0.05 to 2 wt. %, based on total weight of the agent.

The composition can optionally contain at least one protein hydrolyzate and/or one of its derivatives as a care substance of another compound class. The agents contain protein hydrolyzates, for example, in concentrations of 0.01 wt. % to 20 wt. %, preferably 0.05 wt. % to 15 wt. %, and more preferably 0.05 wt. % to 5 wt. %, based on total end-use preparation.

The agent can further have at least one vitamin, one provitamin, one vitamin precursor and/or one of their derivatives as the care substance.

Preferred vitamins, provitamins and vitamin precursors are those normally classified in the groups A, B, C, E, F and H. Retinol (vitamin A₁) as well as 3,4-didehydroretinol, (vitamin A₂) belong in the group of substances designated as vitamin A. The vitamin B group or vitamin B complex include, inter alia, vitamin B₁ (thiamine), vitamin B₂ (riboflavin), vitamin B₃ (nicotinic acid and/or nicotinic acid amide (niacinamide)), vitamin B₅ (pantothenic acid, panthenol and pantolactone), vitamin B₆ (pyridoxine as well as pyridoxamine and pyridoxal). Other vitamin representatives are vitamin C (ascorbic acid), vitamin E (tocopherols, especially a-tocopherol), vitamin F (linoleic acid and/or linolenic acid), and vitamin H.

The agents preferably include vitamins, provitamins and vitamin precursors from groups A, B, C, E and H. Panthenol, pantolactone, pyridoxine and its derivatives, as well as nicotinamide and biotin, are especially preferred. D-panthenol is quite particularly preferably used as a care substance, optionally in combination with at least one of the above-mentioned silicone derivatives.

Thus, if a particularly flexible hold is desired, then the agents can include panthenol instead of or in addition to glycerin and/or propylene glycol. In a preferred embodiment, the agents contain panthenol, preferably in an amount of 0.05 to 10 wt. %, more preferably 0.1 to 5 wt. %, based on total weight of the agent.

Agents according to the invention can also contain at least one plant extract as a care substance. Usually, these extracts are manufactured by extraction of the whole plant. In individual cases, however, it can be preferred to produce the extracts solely from blossoms and/or leaves of the plant. According to the invention, extracts mainly from green tea, oak bark, stinging nettle, hamamelis, hops, henna, chamomile, burdock root, field horsetail, hawthorn, linden flowers, almonds, aloe vera, spruce needles, horse chestnut, sandal wood, juniper, coconut, mango, apricot, lime, wheat, kiwi, melon, orange, grapefruit, sage, rosemary, birch, malva, lady's smock, common yarrow, thyme, lemon balm, rest-harrow, coltsfoot, marshmallow (althaea), meristem, ginseng and ginger are preferred.

The agent can further contain at least one lipid as a care substance. According to the invention, suitable lipids are phospholipids, for example, soy lecithin, egg lecithin and cephalins as well as the substances known under the INCI names Linoleamidopropyl PG-Dimonium Chloride Phosphate, Cocamidopropyl PG-Dimonium Chloride Phosphate and Stearamidopropyl PG-Dimonium Chloride Phosphate. These are commercialized, for example, by the Mona Company under the trade names Phospholipid Phospholipid PTC® and Phospholipid SV®. The agents preferably contain lipids in amounts of 0.01 to 10 wt. %, particularly 0.1 to 5 wt. %, based on total end-use preparation.

Oil bodies are also suitable as a care substance.

Natural and synthetic cosmetic oil bodies include:
vegetal oils. These include sunflower oil, olive oil, soya oil, rapeseed oil, almond oil, jojoba oil, orange oil, wheat germ oil, peach stone oil and the liquid parts of coconut oil. Other triglyceride oils such as the liquid fractions of beef tallow are also suitable, as well as synthetic triglyceride oils, liquid paraffin oils, isoparaffin oils and synthetic hydrocarbons, as well as di-n-alkyl ethers containing a total of 12 to 36 carbon atoms, particularly 12 to 24 carbon atoms such as di-n-octyl ether, di-n-decyl ether, di-n-nonyl ether, di-n-undecyl ether, di-n-dodecyl ether, n-hexyl n-octyl ether, n-octyl n-decyl ether, n-decyl n-undecyl ether, n-undecyl n-dodecyl ether and n-hexyl n-undecyl ether and di-tert.butyl ether, diisopentyl ether, di-3-ethyldecyl ether, tert.butyl n-octyl ether, isopentyl n-octyl ether and 2-methylpentyl n-octyl ether. Commercial products of the compounds 1,3-di-(2-ethylhexyl)cyclohexane (Cetiol® S) and di-n-octyl ether (Cetiol® OE) are preferred.
Ester oils. Ester oils refer to esters of C₆-C₃₀ fatty acids with C₂-C₃₀ fatty alcohols. Monoesters of fatty acids with alcohols having 2 to 24 carbon atoms are preferred. Isopropyl myristate (Rilanit® IPM), isononanoic acid C16-18 alkyl ester (Cetiol® SN), 2-ethylhexyl palmitate (Cegesoft® 24), stearic acid 2-ethylhexyl ester (Cetiol® 868), cetyl oleate, glycerin tricaprylate, cocofatty alcohol caprinate/-caprylate (Cetiol® LC), n-butyl stearate, oleyl erucate (Cetiol® J 600), isopropyl palmitate (Rilanit IPP), oleyl oleate (Cetiol®), lauric acid hexyl ester (Cetiol® A), di-n-butyl adipate (Cetiol® B), myristyl myristate (Cetiol® MM), cetearyl isononanoate (Cetiol® SN), and oleic acid decyl ester (Cetiol® V) are particularly preferred.
Dicarboxylic acid esters such as di-n-butyl adipate, di-(2-ethylhexyl) adipate, di-(2-ethylhexyl) succinate and di-isotridecyl acetate, as well as diol esters such as ethylene glycol dioleate, ethylene glycol di-isotridecanoate, propylene glycol di(2-ethylhexanoate), propylene glycol di-isostearate, propylene glycol di-pelargonate, butane diol di-isostearate, neopentyl glycol dicaprylate;
symmetrical, unsymmetrical or cyclic esters of carbon dioxide with fatty alcohols, as described in DE-OS 197 56 454, glycerin carbonate or dicaprylyl carbonate (Cetiol® CC);
trifatty acid esters of saturated and/or unsaturated linear and/or branched fatty acids with glycerin;
fatty acid partial glycerides, which include monoglycerides, diglycerides and their industrial mixtures. When using industrial products, minor amounts of triglycerides may still be present due to the production process. Partial glycerides preferably comply with Formula (D4-I),

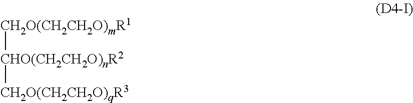

wherein R¹, R² and R³ are, independently of each other, hydrogen or a linear or branched, saturated and/or unsaturated acyl group containing 6 to 22 carbon atoms, preferably 12 to 18 carbon atoms, with the proviso that at least one of these groups is an acyl group and at least one of these groups is hydrogen. The sum of (m+n+q) is 0 or a number from 1 to 100, preferably 0 or 5 to 25. Preferably, R¹ is an acyl group, R² and R³ are hydrogen, and the sum of (m+n+q) is 0. Typical examples are mono- and/or diglycerides based on caproic acid, caprylic acid, 2-ethylhexanoic acid, capric acid, lauric acid, isotridecanoic acid, myristic acid, palmitic acid, palmitoleic acid, stearic acid, isostearic acid, oleic acid, elaidic acid, petroselic acid, linoleic acid, linolenic acid, elaeostearic acid, arachidonic acid, gadoleic acid, behenic acid and erucic acid as well as their industrial mixtures. Oleic acid monoglycerides are preferably used.

The added amount of natural and synthetic cosmetic oil bodies in the agents is typically 0.1 to 30 wt. %, based on total end-use preparation, preferably 0.1 to 20 wt. % and particularly 0.1 to 15 wt. %.

With the addition of a UV filter, both the agent and the treated fibers can be protected against damage from UV radiation. Consequently, at least one UV filter is preferably added to the agent. Suitable UV filters are generally not limited with respect to their structure and physical properties. Indeed, all UV filters that can be used in the cosmetic field having an absorption maximum in the UVA (315-400 nm), UVB (280-315 nm) or UVC (<280 nm) regions are suitable. UV filters having an absorption maximum in the UVB region, especially in the range from about 280 to about 300 nm, are particularly preferred. Preferred UV-filters are chosen from substituted benzophenones, p-aminobenzoates, diphenylacrylates, cinnamates, salicylates, benzimidazoles and o-aminobenzoates.

The agent usually contains UV filters in amounts of 0.01 to 5 wt. %, based on total end-use preparation. Quantities of 0.1-2.5 wt. % are preferred.

In a particular embodiment, the agent further has one or more substantive dyes. Application of the agent then allows the treated keratinic fiber not only to be temporarily styled, but also to be dyed at the same time. This can be particularly desirable when only a temporary dyeing is desired, for example, with flamboyant fashion colors that can be subsequently removed from the keratinic fibers by simply washing them out. Substantive dyes used are typically nitrophenylenediamines, nitroamino phenols, azo dyes, anthraquinones, indophenols or cationic substantive dyes. Particularly preferred cationic substantive dyes are cationic triphenylmethane dyes, such as Basic Blue 7, Basic Blue 26, Basic Violet 2 and Basic Violet 14, aromatic systems substituted with a quaternary nitrogen group, such as Basic Yellow 57, Basic Red 76, Basic Blue 99, Basic Brown 16 and Basic Brown 17, as well as substantive dyes having a heterocycle that exhibits at least one quaternary nitrogen atom. Dyes also known under the names Basic Yellow 87, Basic Orange 31 and Basic Red 51 are particularly preferred cationic substantive dyes.

Cationic substantive dyes commercially available under the trade name Manor® are quite particularly preferred cationic substantive dyes according to the invention. In addition, the compositions can also contain naturally occurring dyestuffs as are present, for example, in henna red, henna neutral, henna black, camomile leaves, sandalwood, black tea, alder buckthorn bark, sage, logwood, madder root, cachou, cedar and alkanet root.

The agents contain substantive dyes preferably in an amount of 0.001 to 20 wt. %, based on total agent.

Preferably, agents according to the invention are exempt from oxidation dye precursors. Oxidation dye precursors are divided into developer components and coupler components. Under the influence of oxidizing agents or from atmospheric oxygen, the developer components form the actual colorants among each other or by coupling with one or more coupler components.

Formulations of the agents can be in all forms typical for styling agents, such as solutions that can be applied as hair water or pump or aerosol spray onto the hair, as well as creams, emulsions, waxes, gels, or surfactant-containing foaming solutions or other preparations suitable for application on the hair.

Agents according to the invention are preferably packaged as a hair cream or hair gel, especially as a hair gel. Hair creams and hair gels preferably also include, in addition to the inventive polymer combination, at least one thickening polymer. This addition is optionally preferred and provides the desired consistency for the agents. Structurants and/or thickening polymers are typically added in amounts of 0.1 to 10 wt. %, based on total product. Quantities of 0.5 to 5 wt. %, particularly 0.5 to 3 wt. %, are preferred.

Polymers can increase the viscosity of aqueous and non-aqueous phases in cosmetic preparations. In aqueous phases, their propensity to increase the viscosity is based on their solubility in water or their hydrophilic nature. They can be used in surface-active and also in emulsion-like systems of the invention.

The agents preferably have at least one polyethylene glycol with a melting point of 20° C. as the structurant. Polyethylene glycols of this type preferably have a molecular weight of 550 g/mol or greater, especially 1000 g/mol or greater.

The agents can include polyethylene glycols in an amount of 0.1 wt. % to 10 wt. %, particularly 0.2 wt. % to 5 wt. %, based on total weight of the agent.

A second subject matter of the invention is the use of the agent for temporary shaping of hair and/or for hair care.

The agents and products containing these agents, especially hair gels or hair creams, give treated hair a very strong, long-lasting hold, while the hair remains flexible. If the agent is a hair gel, then the gel has a pasty consistency that can be uniformly dispersed on the hair without any dripping.

Preferably, the agent of the first subject matter of the invention is a leave-on hair treatment agent.

A third subject matter of the invention is a method for treating keratin-containing fibers, especially human hair, wherein the agent of the first subject matter is applied onto the keratin-containing fibers.

Preferably, the keratin-containing fibers are styled before, during or after application of the agent.

Furthermore, it is preferred according to the method not to rinse out the agent from the keratin-containing fibers.

The following examples are intended to illustrate the subject matter of the present invention in more detail without limiting it in any way.

### EXAMPLES

Unless otherwise stated, the quantities are understood to be in weight percent.

### 1.0 Formulations—

Inventive styling gels A to C according to the following Table were manufactured.

| Raw Materials | A | B | C |
|---|---|---|---|
| 2-Hydroxy-4-methoxybenzophenone-5- | 0.05 | 0.05 | 0.05 |
| sulfonic acid | | | |
| Synthalen ® K¹ | 0.60 | 0.60 | 0.60 |
| Ethanol | 15.00 | 15.00 | 15.00 |
| D-Panthenol | 0.10 | 0.10 | 0.10 |
| Dow Corning 193 Fluid ®² | — | 0.15 | 0.20 |
| Polyethylene glycol | 2.00 | 2.10 | — |
| Aquastyle ® 300³ | 3.50 | 3.30 | 6.60 |
| Fixate ® Superhold⁴ | 3.50 | 3.30 | 3.30 |
| PEG-40 hydrogenated castor oil | 0.60 | 0.60 | 0.60 |
| 2-Amino-2-methylpropanol | 0.75 | 0.70 | 0.70 |
| Perfume | 0.20 | 0.20 | 0.20 |

| | |
|---|---|
| Water | ad 100 |

¹Polyacrylic acid (ca. 89% active substance content; INCI name: Carbomer) (3V Sigma)
²Silicone-glycol copolymer (INCI name: PEG-12 Dimethicone) (Dow Corning)
³Copolymet of N-vinyl pyrrolidone/N-vinyl caprolactam/N-(3-dimethylaminopropyl)methacrylamide and 3-(methacryloylamino)propyl-lauryl-dimethylammonium chloride (active substance 30 wt. % in water/ethanol, INCI name: Polyquaternium-69) (ISP),
⁴Active substance content 30 wt. %, INCI name: Polyacrylate-2 Crosspolymer (Noveon)

All of gels A to C were not sticky and exhibited a pasty rheology as a plastic fluid. In spite of the pasty theology, the gels were excellently dispersed on the hair (see below).

Each of the formulations A to C was tested on the hair of a test person. For this, an adequate amount of gel was placed onto the palm of the hand and dispersed in the hair. The hair was then dressed and styled. The resulting hairstyle had a perfect hold without any sticky residues and the hair was cared for.

We claim:

1. Agent for treating keratin-containing fibers comprising in a cosmetically acceptable carrier:
(a) at least one amphiphilic, cationic polymer having at least one structural unit of Formula (I), at least one structural unit of Formula (II), at least one structural unit of Formula (III), and at least one structural unit of Formula (IV), 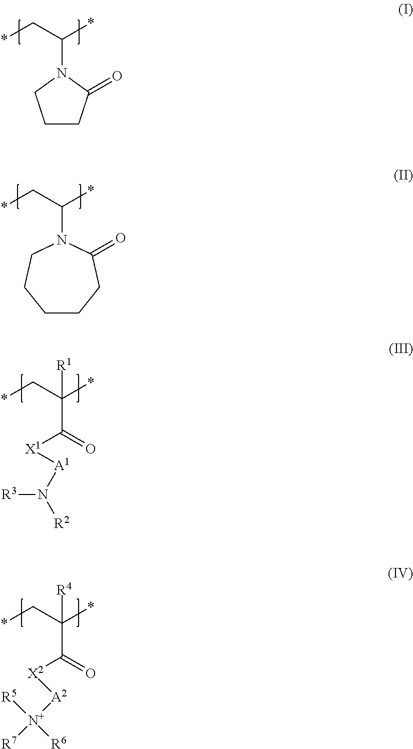
wherein
R¹ and R⁴ are, independently of one another, stand for a hydrogen atom or a methyl group,
X¹ and X² are, independently of one another, stand for an oxygen atom or an NH group,
A¹ and A² stand independently of one another for an ethane-1,2-diyl, propane-1,3-diyl or butane-1,4-diyl group,
R², R³, R⁵ and R⁶ stand independently of one another for a (C₁ to C₄) alkyl group,
R⁷ is a (C₈ to C₃₀) alkyl group, and
(b) at least one polymer having one or more silicone-containing side chains and one or more anionic groups.

2. Agent according to claim 1 wherein A¹ and A² are, independently of one another, ethane-1,2-diyl or propane-1,3-diyl.

3. Agent according to claim 1 wherein R², R³, R⁵ and R⁶ are, independently of one another, methyl or ethyl.

4. Agent according to claim 1 wherein R⁷ is decyl, undecyl, dodecyl, tridecyl, tetradecyl, hexadecyl, octadecyl, eicosyl or docosyl.

5. Agent according to claim 1 wherein the at least one amphiphilic, cationic polymer is chosen from at least one polymer having at least one structural unit of Formula (I), at least one structural unit of Formula (II), at least one structural unit of Formula (III-8), and at least one structural unit of Formula (IV-8) 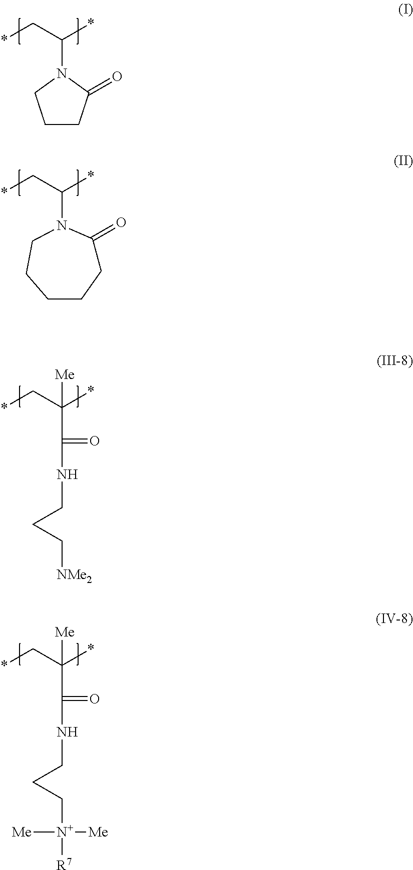
wherein R⁷ is a (C₈ to C₃₀) alkyl group.

6. Agent according to claim 1 wherein the at least one amphiphilic, cationic polymer is present in an amount of 0.05 wt. % to 10.0 wt. %, based on total weight of the agent.

7. Agent according to claim 1 wherein the at least one polymer with one or more silicone-containing side chains and one or more anionic groups is present in an amount of 0.05 wt. % to 10 wt. %, based on total weight of the agent.

8. Agent according to claim 1 wherein the at least one polymer with one or more silicone-containing side chains and one or more anionic groups is chosen from at least one polymer obtained by copolymerizing the following monomers (i) and optionally (ii):
(i) at least one monomer having an olefinic unsaturated group, a silicone group, and, when no optional monomer (ii) is added, at least one anionic group, and
(ii) optionally at least one additional monomer chosen from non-ionic monomers, anionic monomers, cationic monomers, associative monomers or crosslinking monomers.

9. Agent according to claim 1 wherein the polymer with one or more silicone-containing side chains and one or more anionic groups is chosen from at least one polymer obtained by copolymerizing at least the following monomers (i) to (v):
(i) at least one monomer having an olefinic unsaturated group and a silicone group,
(ii) at least one additional monomer chosen from (preferably olefinic unsaturated) anionic monomers,
(iii) at least one additional non-ionic monomer,
(iv) optionally, at least one additional associative monomer, and
(v) optionally, at least one additional crosslinking monomer.

10. Agent according to claim 1 wherein the polymer with at least one silicone-containing side chain and at least one anionic group is at least one polymer obtained by copolymerizing at least the following monomers (i) to (v):
(i) at least one monomer that carries an olefinic unsaturated group and a silicone group, and
(ii) acrylic acid and/or methacrylic acid,
(iii) at least one additional non-ionic monomer chosen from (C₁ to C₄) alkyl esters of acrylic acid and (C₁ to C₄) alkyl esters of methacrylic acid,
(iv) optionally, at least one additional associative monomer, and
(v) optionally, at least one additional crosslinking monomer.

11. Agent according to claim 8 wherein at least one monomer (i) is chosen from compounds according to Formulae (A1) and (A2), 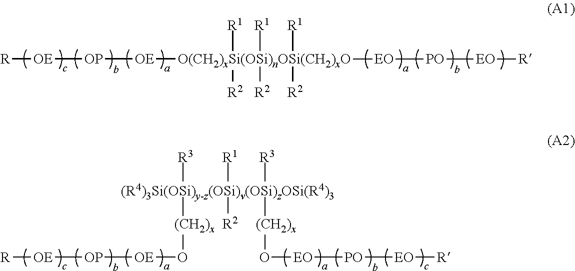
wherein
R¹ and R² are, independently of one another, a (C₁ to C₃₀) alkyl group, a (C₁ to C₂₀) alkyl group substituted with at least one halogen atom, a (C₃ to C₈) cycloalkyl group, a (C₆ to C₁₄) aryl group or (C₂ to C₂₀) alkenyl group,
R³ is a (C₁ to C₃₀) alkyl group, a (C₁ to C₂₀) alkyl group substituted with at least one halogen atom, a (C₃ to C₈) cycloalkyl group, a (C₆ to C₁₄) aryl group or (C₂ to C₂₀) alkenyl group,
R⁴ are each independently a (C₁ to C₃₀) alkyl group, a (C₆ to C₁₄) aryl group, a (C₂ to C₂₀) alkenyl group,
at least one of R and R′ is an ester group of an olefinic unsaturated carboxylic acid according to one of Formulae (Est1) to (Est7), wherein the other constituent is an ester group of one of Formulae (Est1) to (Est7) or is a hydrogen atom, 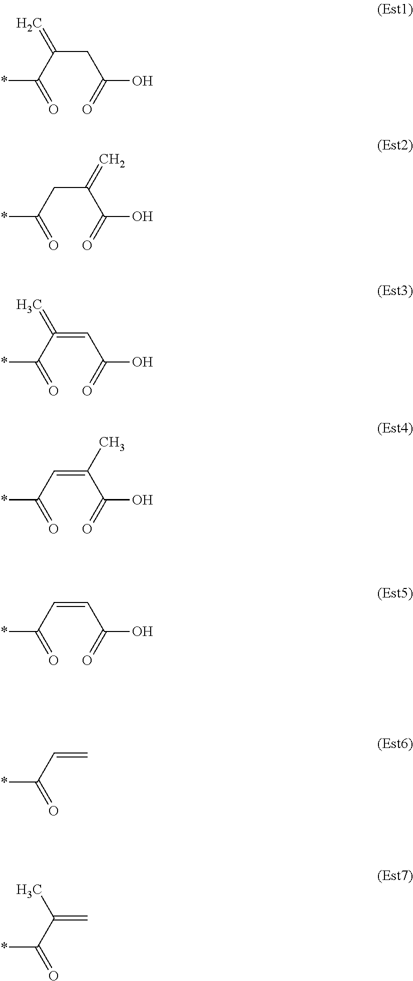
E is an ethane-1,2-yl group,
P is a propane-1,2-diyl group or a propane-1,3-diyl group,
a, b and c are, independently of one another, a number from 0 to 100,
n is a number from 0 to 1000
x is a number 2 or 3,
v is a number from 0 to 200,
y is a number from 1 to 200, and
z is less than or equal to y.

12. Agent according to claim 1 further comprising a film-forming and/or setting polymer.

13. Agent according to claim 12 wherein the film-forming and/or setting polymers are present in an amount of 0.01 wt. % to 20 wt. %, based on total weight of the agent.

14. Hair cream or hair gel comprising the agent according to claim 1.
